SEQUENCE LISTING (53 sequences)

SEQ ID NO: 1 - DNA, 375 bp, Artificial Sequence
  3B9 VL1-VH nucleic acid sequence
caggtgcagctggtgcagtctggggctgaggtgaagaagcctgggtcctcggtgaaggtctcctgcaaggcttctggaggcaccttcagcagctatgctatcagctgggtgcgccaggcccctggacaagggcttgagtggatgggagggatcatccctctctttgatacaacaaagtacgcacagcagttccagggcagagtcacgattaccgcggacgaatccacgagcacagcctacatggagctgagcagcctgagatctgaggacacggccgtattttactgtgcgagagatcgggatattttgactgattattatcccatgggcggtatggacgtctggggccaagggaccacggtcaccgtctcctca

SEQ ID NO: 2 - protein, 125 aa, Artificial Sequence
  3B9 VL1-VH amino acid sequence
QVQLVQSGAEVKKPGSSVKVSCKASGGTFSSYAISWVRQAPGQGLEWMGGIIPLFDTTKYAQQFQGRVTITADESTSTAYMELSSLRSEDTAVFYCARDRDILTDYYPMGGMDVWGQGTTVTVSS

SEQ ID NO: 3 - DNA, 324 bp, Artificial Sequence
  39B9 VL1-VL nucleic acid sequence
gccatccagttgacccagtctccatcctccctgtctgcatctgtaggagacagagtcaccatcacttgccgggcaagtcagggcattagcagtgttttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtctaatagttacccgctcactttcggcggagggaccaaggtggagatcaaacgt

SEQ ID NO: 4 - protein, 108 aa, Artificial Sequence
  39B9 VL1-VL amino acid sequence
AIQLTQSPSSLSASVGDRVTITCRASQGISSVLAWYQQKPGKAPKLLIYDASSLESGVPSRFSGSGSGTDFTLTISSLQPEDFATYYCQQSNSYPLTFGGGTKVEIKR

SEQ ID NO: 5 - DNA, 324 bp, Artificial Sequence
  39B9 VL5-VL nucleic acid sequence
gacatcctgatgacccagtctccatcctccctgtctgcatctgtaggagacagagtcaccatcacttgtcgggcgagtcaggatattagcagctggttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtctaatagttacccgctcactttcggcggagggaccaaggtggagatcaaacga

SEQ ID NO: 6 - protein, 108 aa, Artificial Sequence
  39B9 VL5-VL amino acid sequence
DILMTQSPSSLSASVGDRVTITCRASQDISSWLAWYQQKPGKAPKLLIYDASSLESGVPSRFSGSGSGTDFTLTISSLQPEDFATYYCQQSNSYPLTFGGGTKVEIKR

SEQ ID NO: 7 - DNA, 357 bp, Artificial Sequence
  12A VH nucleic acid sequence
caggtgcagctggtggagtcttggggaggcgtggtccagcctgggaggtccctgagactctcctgtgcagcgtctggattcaccttcagtaactatgacatgtactgggtccgccaggctccaggcaaggggctggagtgggtggcagttatattagatgatggaaataataattactacgcagactccgtgaagggccgattcaccatctccagagacaattccaagaaaaaggtgtatctgcaaatgaatagcctgagagctgaggacacggctgtgtattactgtgtgagagcgtcccctaactggggtcttcttgacttctggggccagggaaccctggtcaccgtctcgagt

SEQ ID NO: 8 - protein, 119 aa, Artificial Sequence
  12A VH amino acid sequence
QVQLVESWGGVVQPGRSLRLSCAASGFTFSNYDMYWVRQAPGKGLEWVAVILDDGNNNYYADSVKGRFTISRDNSKKKVYLQMNSLRAEDTAVYYCVRASPNWGLLDFWGQGTLVTVSS

SEQ ID NO: 9 - DNA, 324 bp, Artificial Sequence
  12A VL nucleic acid sequence
gaaattgtgttgacacagtctccatcctcactgtctgcatctgtaggagacagagtcaccatcacttgtcgggcgagtcagggtattagcagctggttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgatcaccttcggccaagggacacgactggagattaaacgt

SEQ ID NO: 10 - protein, 108 aa, Artificial Sequence
  12A VL amino acid sequence
EIVLTQSPSSLSASVGDRVTITCRASQGISSWLAWYQQKPGKAPKLLIYDASSLESGVPSRFSGSGSGTDFTLTISSLQPEDFATYYCQQFNSYPITFGQGTRLEIKR

SEQ ID NO: 11 - DNA, 357 bp, Artificial Sequence
  5C VH nucleic acid sequence
caggtgcagctggtgcagtctgggggaggcgtggtccagcctgggaggtccctgagactctcctgtgcagcctctggattcatcttcagtagctatgacatgtactgggtccgccaggctccaggcaaggggctggagtgggtggcagttatattatatgatggaaataataaatactacgcagactccgtgaagggccgattcaccatctccagagacaattccaagaacacggtgtatctgcaaatgaacagcctgagagctgaggacacggctgtgtattactgtgtgagagcgtcccctaactggggtctttttgacttctggggccagggaaccctggtcaccgtctcgagt

SEQ ID NO: 12 - protein, 119 aa, Artificial Sequence
  5C VH amino acid sequence
QVQLVQSGGGVVQPGRSLRLSCAASGFIFSSYDMYWVRQAPGKGLEWVAVILYDGNNKYYADSVKGRFTISRDNSKNTVYLQMNSLRAEDTAVYYCVRASPNWGLFDFWGQGTLVTVSS

SEQ ID NO: 13 - DNA, 324 bp, Artificial Sequence
  5C VL nucleic acid sequence
gacatccagatgacccagtctccatcctccctgtctgcatctgtaggagacagagtcaccatcacttgccgggcaagtcagggcattagcagtgatttagcctggtatcagcagaaaccagggaaagctcctaagctcctgatgtatgatgcctccagtttggaaagtggggtcccatcaaggttcagcggcagtggatctgggacagatttcactctcaccatcagcagcctgcagcctgaagattttgcaacttattactgtcaacagtttaatagttacccgatcaccttcggccaagggacacgactggagattaaacgt

SEQ ID NO: 14 - protein, 108 aa, Artificial Sequence
  5C VL amino acid sequence
DIQMTQSPSSLSASVGDRVTITCRASQGISSDLAWYQQKPGKAPKLLMYDASSLESGVPSRFSGSGSGTDFTLTISSLQPEDFATYYCQQFNSYPITFGQGTRLEIKR

SEQ ID NO: 15 - protein, 5 aa, Artificial Sequence
  39B9 VH CDR1
SYAIS

SEQ ID NO: 16 - protein, 17 aa, Artificial Sequence
  39B9 VH CDR2
GIIPLFDTTKYAQQFQG

SEQ ID NO: 17 - protein, 19 aa, Artificial Sequence
  39B9 VH CDR3
CARDRDILTDYYPMGGMDV

SEQ ID NO: 18 - protein, 5 aa, Artificial Sequence
  12A VH CDR1
NYDMY

SEQ ID NO: 19 - protein, 17 aa, Artificial Sequence
  12A VH CDR2
VILDDGNNNYYADSVKG

SEQ ID NO: 20 - protein, 13 aa, Artificial Sequence
  12A VH CDR3
CVRASPNWGLLDF

SEQ ID NO: 21 - protein, 5 aa, Artificial Sequence
  5C VH CDR1
SYDMY

SEQ ID NO: 22 - protein, 17 aa, Artificial Sequence
  5C VH CDR2
VILYDGNNKYYADSVKG

SEQ ID NO: 23 - protein, 13 aa, Artificial Sequence
  5C VH CDR3
CVRASPNWGLFDF

SEQ ID NO: 24 - protein, 11 aa, Artificial Sequence
  39B9 VL1 VL CDR1
RASQGISSVLA

SEQ ID NO: 25 - protein, 7 aa, Artificial Sequence
  IL-6 antibody VL CDR2
DASSLES

SEQ ID NO: 26 - protein, 9 aa, Artificial Sequence
  39B9 VL1 VL CDR3
QQSNSYPLT

SEQ ID NO: 27 - protein, 11 aa, Artificial Sequence
  39B9 VL5 VL CDR1
RASQDISSWLA

SEQ ID NO: 28 - protein, 11 aa, Artificial Sequence
  12A VL CDR1
RASQGISSWLA

SEQ ID NO: 29 - protein, 9 aa, Artificial Sequence
  12A VL CDR3
QQSNSYPIT

SEQ ID NO: 30 - protein, 11 aa, Artificial Sequence
  5C VL CDR1
RASQGISSVDA

SEQ ID NO: 31 - protein, 9 aa, Artificial Sequence
  VL CDR3 motif
  MISC_FEATURE (4)..(4): Xaa at position 4 is asparagine or glutamine
QQSXSYPLT

SEQ ID NO: 32 - protein, 17 aa, Artificial Sequence
  VH CDR2 motif
  MISC_FEATURE (5)..(5): Xaa at position 5 can be leucine or alanine
  MISC_FEATURE (7)..(7): Xaa at position 7 can be aspartic acid or glutamic acid
  MISC_FEATURE (14)..(14): Xaa at position 14 can be glutamine or lysine
GIIPXFXTTKYAQXFQG

SEQ ID NO: 33 - protein, 16 aa, Artificial Sequence
  VH CDR3 motif
  MISC_FEATURE (11)..(11): Xaa at position 11 is methionine or leucine
DRDILTDYYPXGGMDV

SEQ ID NO: 34 - protein, 8 aa, Artificial Sequence
  FRW3 mofitf
  MISC_FEATURE (4)..(4): Xaa at position 4 is phenylalanine or tyrosine
TAVXYCAR

SEQ ID NO: 35 - protein, 16 aa, Artificial Sequence
  NI-1201-WT VH CDR3
DRDILTDYYPMGGMDV

SEQ ID NO: 36 - protein, 8 aa, Artificial Sequence
  NI-1201-WT FRW3
TAVFYCAR

SEQ ID NO: 37 - protein, 17 aa, Artificial Sequence
  NI-1201-A VL CDR3
GIIPLFDTTKYAQKFQG

SEQ ID NO: 38 - protein, 8 aa, Artificial Sequence [sequence omitted]

SEQ ID NO: 39 - protein, 16 aa, Artificial Sequence
  NI-1201-B VH CDR3
DRDILTDYYPLGGMDV

SEQ ID NO: 40 - protein, 17 aa, Artificial Sequence
  NI-1201-C VH CDR2
GIIPAFETTKYAQKFQG

SEQ ID NO: 41 - protein, 9 aa, Artificial Sequence
  NI-1201-D VL CDR3
QQSQSYPLT

SEQ ID NO: 42 - protein, 5 aa, Artificial Sequence
  anti-CD3 CDRH1
GYGMH

SEQ ID NO: 43 - protein, 17 aa, Artificial Sequence
  anti-CD3 CDRH2
VIWYDGSKKYYVDSVKG

SEQ ID NO: 44 - protein, 9 aa, Artificial Sequence
  anti-CD3 CDRH3
QMGYWHFDL

SEQ ID NO: 45 - protein, 11 aa, Artificial Sequence
  anti-CD3 CDRL1
RASQSVSSYLA

SEQ ID NO: 46 - protein, 7 aa, Artificial Sequence
  anti-CD3 CDRL2
DASNRAT

SEQ ID NO: 47 - protein, 10 aa, Artificial Sequence
  anti-CD3 CDRL3
QQRSNWPPLT

SEQ ID NO: 48 - protein, 118 aa, Artificial Sequence [sequence omitted]

SEQ ID NO: 49 - protein, 108 aa, Artificial Sequence [sequence omitted]

SEQ ID NO: 50 - protein, 448 aa, Artificial Sequence
  anti-CD3 HC
QVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 215 aa, Artificial Sequence
  anti-CD3 LC
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 454 aa, Artificial Sequence
  TZLS-501 heavy chain
QVQLVQSGAEVKKPGSSVKVSCKASGGTFSSYAISWVRQAPGQGLEWMGGIIPLFDTTKYAQKFQGRVTITADESTSTAYMELSSLRSEDTAVYYCARDRDILTDYYPMGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 214 aa, Artificial Sequence
  TZLS-501 light chain
AIQLTQSPSSLSASVGDRVTITCRASQGISSVLAWYQQKPGKAPKLLIYDASSLESGVPSRFSGSGSGTDFTLTISSLQPEDFATYYCQQSNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0190804 A1
(43) Pub. Date: Jun. 22, 2023

(54) COMPOSITIONS AND METHODS FOR AUGMENTING CHIMERIC ANTIGEN RECEPTOR (CAR) T CELL THERAPIES

(71) Applicant: Tiziana Life Sciences PLC, London (GB)

(72) Inventor: Kunwar SHAILUBHAI, Line Lexington, PA (US)

(21) Appl. No.: 17/927,460

(22) PCT Filed: Jun. 17, 2021
(86) PCT No.: PCT/EP2021/066469

§ 371 Date: Nov. 23, 2022

Related U.S. Application Data
(60) Provisional application No. 63/058,783, filed on Jul. 30, 2020.
(60) Provisional application No. 63/040,101, filed on Jun. 17, 2020.

(51) Int. Cl. A61K 35/17; C07K 16/28; C12N 5/0775

(52) U.S. Cl. CPC A61K 35/17; C07K 16/2809; C12N 5/0662; A61K 2039/505

(57) ABSTRACT

This disclosure relates to improving CAR-T expansion and/or survival using CD-3 antibodies alone or in combination with other co-stimulatory molecules such as an anti-IL-6 receptor monoclonal antibody, an anti-CD28 monoclonal antibody, an IL-17 monoclonal antibody or specific inhibitors of signaling pathways of phosphatidylinositol 3-kinase (PI3K), protein kinase B (AKT), or mammalian target of rapamycin (mTOR).

### CROSS REFERENCE TO RELATED APPLICATIONS

This application claims the benefit of priority to U.S. Provisional Pat. Application Serial No. 63/040,101 filed on Jun. 17, 2020 and U.S. Provisional Patent Application Serial No. 63/058,783 filed on Jul. 30, 2020, the contents of which are hereby incorporated by reference in their entireties.

### REFERENCE TO SEQUENCE LISTING

This application is being filed electronically via EFS-Web and includes an electronically submitted sequence listing in .txt format. The .txt file contains a sequence listing entitled “TIZI_027_001WO_SeqList_ST25.txt” created on Jun. 16, 2021 and having a size of 33 kilobytes. The sequence listing contained in this .txt file is part of the specification and is incorporated herein by reference in its entirety.

### FIELD OF THE INVENTION

This disclosure relates to improving cell therapies, CAR-T expansion and/or survival using CD-3 antibodies alone or in combination with other co-stimulatory molecules such as an anti-IL-6 receptor monoclonal antibody, an anti-CD28 monoclonal antibody or specific inhibitors of signaling pathways of phosphatidylinositol 3-kinase (PI3K), protein kinase B (AKT), or mammalian target of rapamycin (mTOR).

### BACKGROUND

Chimeric antigen receptor T-cells (CAR-T), while utilizing the expansion and killing effects of cytotoxic T cells, specifically improve function of T cells and avoid issues common to T cell therapy such as the dependence on human leukocyte antigen (HLA) interactions for activation of T cell effector functions. HLA is often down regulated in cancer cells to promote immune system escape, which makes it more difficult for engineered T cells to activate an effector response. The expression of CAR allows the T cell to reduce its dependence on HLA mediated activation and elicit an effector response by binding to tumor specific surface proteins. The basic CAR consists of an antigen binding domain which is a single chain variable region antibody fragment (scFV), an extracellular domain, transmembrane domain and intracellular signaling domains. The intracellular domain is typically the CD3 ζ chain that is typically associated with the T cell receptor (TCR) complex. CAR-T cells combine the dynamic of T cells with the antigen-specificity of an antibody and they can bind the tumor antigen without antigen processing and independent of HLA-mediated antigen presentation.

CAR-T cell therapy has been successful in the treatment of B-cell leukemias, most notably acute B-cell lymphoblastic leukemia (B-cell ALL) treated with anti-CD19 CAR-T cells.

Despite encouraging clinical outcomes, relapse rates following CAR-T therapy occur, which limits the utility of this promising treatment approach. Therefore, it is critical to address the current limitations of CAR-T cell therapy. The present disclosure provides aspects and embodiments of compositions and methods to fill this unmet need.

### SUMMARY

In one aspect, the disclosure provides a method of improving cell expansion and/or survival comprising contacting a cell with a composition comprising an anti-CD3 antibody. In some embodiments, the cell is an engineered cell. In some embodiments, the contacting is ex vivo, in vivo or both.

In some embodiments, the cell is a lymphocyte. In some embodiments, the lymphocyte is a B-cell or a T-cell. In some embodiments, the T cell is a CAR-T cell. In some embodiments, the cell is a stem cell. In some embodiments, the stem cell is a human embryonic stem cell, a tissue-specific stem cell, a neural stem cell, a mesenchymal stem cell, a hematopoietic stem cells, an induced pluripotent stem cell, an epidermal stem cell, an epithelial stem cell, and/or a neural stem cell.

In one aspect, the disclosure provides a method of enhancing cell therapy in a subject comprising administering to a subject in need thereof a composition comprising an anti-CD3 antibody. In some embodiments, the cell therapy is CAR-T cell therapy. In some embodiments, the cell therapy is stem cell therapy. In some embodiments, the stem cell is a human embryonic stem cell, a tissue-specific stem cell, a neural stem cell, a mesenchymal stem cell, a hematopoietic stem cells, an induced pluripotent stem cell, an epidermal stem cell, an epithelial stem cell, and/or a neural stem cell.

In some embodiments, the composition comprising an anti-CD3 antibody improves the clinical outcome of the cell therapy. In some embodiments, the anti-CD3 antibody is a monoclonal antibody, a bispecific antibody or a trispecific antibody.

In some embodiments, the bispecific antibody has specificity for CD3 and IL-6R, CD28 or TNF.

In some embodiments, the trispecific antibody has specificity for: i) CD3, IL6R, and CD28; ii) CD3, IL6R and TNF; iii) CD3, CD28 and TNF; or iv) IL-6, IL-17.

In some embodiments, the composition comprising an anti-CD3 antibody further include one or more co-stimulatory agents. In some embodiments, the co-stimulatory agent is a CD28 antibody, an IL-6R antibody, a PI3K inhibitor, an Akt inhibitor or a mTor inhibitor. In some embodiments, the CD28 antibody is a monoclonal antibody, a bispecific antibody or a trispecific antibody.

In some embodiments, the bispecific antibody has specificity for CD28 and IL-6R or TNF.

In some embodiments, the trispecific antibody has specificity for CD28, IL-6R, and TNF.

In some embodiments, the IL-6R antibody is a monoclonal antibody, a bispecific antibody or a trispecific antibody. In some embodiments, the bispecific antibody has specificity for IL-6R and CD28 or TNF. In some embodiments, the trispecific antibody has specificity for IL-6R, CD28, and TNF.

In some embodiments, the CD3 antibody and or the CD28 antibody are coated on macroporous beads. In some embodiments, the CD3 antibody is administered nasally, orally, subcutaneously, intravenously or by inhalation.

In some embodiments, the CD3 antibody comprises a heavy chain complementarity determining region 1 (CDRH1) comprising the amino acid sequence GYGMH (SEQ ID NO: 42), a heavy chain complementarity determining region 2 (CDRH2) comprising the amino acid sequence VIWYDGSKKYYVDSVKG (SEQ ID NO: 43), a heavy chain complementarity determining region 3 (CDRH3) comprising the amino acid sequence QMGYWHFDL (SEQ ID NO: 44), a light chain complementarity determining region 1 (CDRL1) comprising the amino acid sequence RASQSVSSYLA (SEQ ID NO: 45), a light chain complementarity determining region 2 (CDRL2) comprising the amino acid sequence DASNRAT (SEQ ID NO: 46), and a light chain complementarity determining region 3 (CDRL3) comprising the amino acid sequence QQRSNWPPLT (SEQ ID NO: 47).

In some embodiments, the CD3 antibody comprises a variable heavy chain amino acid sequence comprising the amino acid sequence of SEQ ID NO: 48 and a variable light chain amino acid sequence comprising the amino acid sequence of SEQ ID NO: 49. In some embodiments, the CD3 antibody comprises a comprising a heavy chain amino acid sequence comprising the amino acid sequence of SEQ ID NO: 50 and a light chain amino acid sequence comprising the amino acid sequence of SEQ ID NO: 51.

In some embodiments, the IL-6R antibody comprising a VH CDR1 region comprising the amino acid sequence of SEQ ID NO: 15, a VH CDR2 region comprising the amino acid sequence of SEQ ID NO: 37, a VH CDR3 region comprising the amino acid sequence of SEQ ID NO: 35, a VL CDR1 region comprising the amino acid sequence of SEQ ID NO: 24, a VL CDR2 region comprising the amino acid sequence of SEQ ID NO: 25, and a VL CDR3 region comprising the amino acid sequence of SEQ ID NO: 26. In some embodiments, the IL-6R antibody is tocilizumab or sarilumab.

In some embodiments, the anti-CD3 antibody is administered prior to, subsequent to, both prior and subsequence to, and/or simultaneously with administration of a cell therapy cell composition to the subject. In some embodiments, the anti-CD3 antibody is administered 24 to 48 hours prior to administering the cell therapy composition to the subject. In some embodiments, the anti-CD3 antibody is administered 14 to 21 days after administering the cell therapy cell composition.

In some embodiments, administering an anti-CD3 antibody prior to administering the cell therapy composition results in lymphodepletion and/or immunosuppression.

In some embodiments, the anti-CD3 antibody is formulated in a pharmaceutical composition comprising the anti-CD3 antibody and a pharmaceutically acceptable carrier. In some embodiments, the pharmaceutical composition comprises a unit dose of 0.5 mg, 1.0 mg, 1.5 mg, 2.0 mg, 2.5 mg, 3.0 mg, or 4 mg of anti-CD3 antibody..

### BRIEF DESCRIPTION OF THE DRAWINGS

FIG. 1 is an illustration depicting the methods of the disclosure. Specifically engineering CAR-T cells to kill tumor cells in cancer patients. The process of ex vivo and in vivo expansion of CAR-T cells can be improved by co-stimulation with anti-CD3/anti-CD28 and small molecular inhibitors of PI-3K/Akt/mTOR axis. Anti-IL-6 receptor mAb may also enhance survival of CAR-T cells.

FIG. 2 is a plot showing the pharmacokinetic profile of foralumab administered subcutaneously or intravenously in a mouse model.

FIG. 3 is a table showing the pharmacokinetic profile of foralumab administered intravenously using different dosing regimens in human subjects.

FIG. 4 is a table showing the apparent Cmax and AUC values of foralumab administered intravenously using different dosing regimens in human subjects.

FIG. 5 is a set of plots showing the time course levels of foralumab in blood plasma following intravenous administration in a human subject.

FIG. 6 is a set of plot showing the time course levels of foralumab in blood plasma and CD3 modulation following intravenous administration in a human subject.

FIG. 7 is a set of plots showing the levels of CD3 modulation on CD4+ and CD8+ T cells over time following intravenous administration of foralumab at different dosages in human subjects.

FIG. 8 is a plot showing levels of CD3 modulation on CD4+ and CD8+ T cells over time following intravenous administration of foralumab at different dosages in human subjects.

FIG. 9 is a table showing the mean TCR-CD3 modulation on CD45+ lymphocytes at different time points following intravenous administration of foralumab at different dosages in human subjects.

FIG. 10 is a table showing the mean peak of cytokine concentrations following intravenous administration of foralumab at different dosages in human subjects.

FIG. 11 is a table showing the summary tabulation of all serious adverse events in human subjects following foralumab administered intravenously.

FIG. 12 is a table showing a listing of reported infusion related reactions (IRRs) in individual human subjects following intravenous administration of foralumab.

FIG. 13 is a table showing a listing of reported infusion related reactions (IRRs) in individual human subjects following intravenous administration of foralumab.

FIG. 14 is a table showing a summary of liver function test abnormalities in human subjects following intravenous administration of foralumab.

FIGS. 15A-15C are schematics showing illustrative embodiments of a clinical dosing regimen for administration of foralumab for enhancing CAR-T cell therapy.

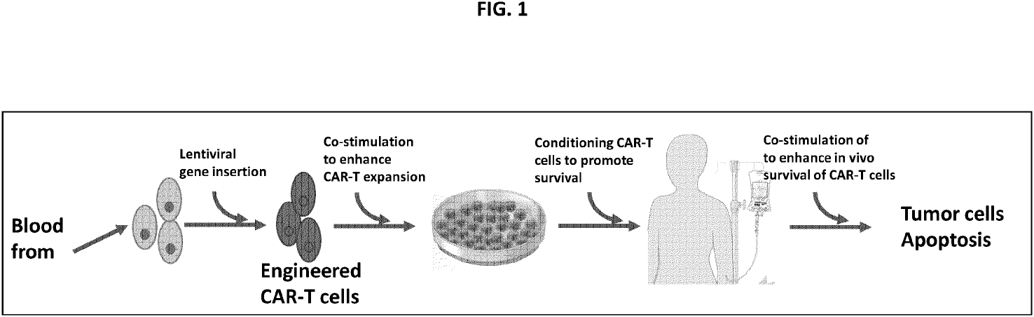

### DETAILED DESCRIPTION

The present disclosure provides compositions and methods for improving cell therapy such as chimeric antigen receptor T-cell therapy (CAR-T cell therapy), CAR-T therapy represents a promising new therapeutic option for patients with a variety of hematological and solid cancers and potentially for patients with autoimmune diseases. However, the complex manufacturing processes and poor CAR-T expansion and survival in ex vivo processes are cost prohibitive. An improved CAR-T therapy can be achieved through more efficient production processes by optimizing the ex vivo expansion conditions and or providing concomitant therapies to improve clinical benefit of CAR-T cells. Accordingly, the present disclosure provides composition and for increasing ex vivo expansion of CART-T cells as well as in vivo treatment regimens. Specifically, the composition of and methods of the present disclosure utilizes anti-CD3 monoclonal antibodies, either alone or in combination with other co-stimulatory molecules such as an anti-IL-6 receptor (IL-6R) monoclonal antibody, an anti-CD28 monoclonal antibody or specific inhibitors of signaling pathways of phosphatidylinositol 3-kinase (PI3K), protein kinase B (AKT), or mammalian target of rapamycin (mTOR). More specifically, intravenous or subcutaneous administration of foralumab, a fully human anti-CD3 monoclonal antibody may be used as a lymphodepletion agent to improve survival of CAR-T cells. The use of foralumab for enhanced lymphodepletion to replace other lymphodepleting agents such as cyclophosphamide/fludarabine (cy/flu) could be administered at different stages of pre and post CAR-T therapy either alone or in combination with other co-stimulatory molecules such anti-IL-6, anti-IL-17 or other antiinflammatory drugs.

### Anti-CD3 Antibodies

Antibodies specific for CD3 epsilon chain (CD3ε) and antigen binding fragments thereof are referred to herein as an “anti-CD3 antibody” or “CD3 antibody”, and the compositions are referred to herein as an “anti-CD3 antibody compositions.” Any anti-CD3 antibody known in the art is suitable for use in the present disclosure. The anti-CD3 antibody is a monoclonal antibody.

The anti-CD3 antibodies can be any antibodies specific for CD3. The anti-CD3 antibody can be a polyclonal, monoclonal, recombinant, e.g., a chimeric, de-immunized or humanized, fully human, non-human, e.g., murine, single chain antibody or single domain antibody. In some embodiments the antibody has effector function and can fix complement. In some embodiments, the antibody has reduced or no ability to bind an Fc receptor. For example, the anti-CD3 antibody can be an isotype or subtype, fragment or other mutant, which does not support binding to an Fc receptor, e.g., it has a mutagenized or deleted Fc receptor binding region. The antibody can be coupled to a toxin or imaging agent.

A number of anti-CD3 antibodies are known, including but not limited to OKT3 (muromonab/Orthoclone OKT3.TM., Ortho Biotech, Raritan, N.J.; U.S. Pat. No. 4,361,549); hOKT3(1 (Herold et al., N.E.J.M. 346(22):1692-1698 (2002); HuM291 (Nuvion.TM., Protein Design Labs, Fremont, Calif.); gOKT3-5 (Alegre et al., J. Immunol. 148(11):3461-8 (1992); 1F4 (Tanaka et al., J. Immunol. 142:2791-2795 (1989)); G4.18 (Nicolls et al., Transplantation 55:459-468 (1993)); 145-2C11 (Davignon et al., J. Immunol. 141(6):1848-54 (1988)); and as described in Frenken et al., Transplantation 51(4):881-7 (1991); U.S. Pat. Nos. 6,491,9116, 6,406,696, and 6,143,297).

Methods for making such antibodies are also known. A full-length CD3 protein or antigenic peptide fragment of CD3 can be used as an immunogen, or can be used to identify anti-CD3 antibodies made with other immunogens, e.g., cells, membrane preparations, and the like, e.g., E rosette positive purified normal human peripheral T cells, as described in U.S. Pat. Nos. 4,361,549 and 4,654,210. The anti-CD3 antibody can bind an epitope on any domain or region on CD3.

Chimeric, humanized, de-immunized, or completely human antibodies are desirable for applications which include repeated administration, e.g., therapeutic treatment of human subjects.

Chimeric antibodies contain portions of two different antibodies, typically of two different species. Generally, such antibodies contain human constant regions and variable regions from another species, e.g., murine variable regions. For example, mouse/human chimeric antibodies have been reported which exhibit binding characteristics of the parental mouse antibody, and effector functions associated with the human constant region. See, e.g., Cabilly et al., U.S. Pat. No. 4,816,567; Shoemaker et al., U.S. Pat. No. 4,978,745; Beavers et al., U.S. Pat. No. 4,975,369; and Boss et al., U.S. Pat. No. 4,816,397, all of which are incorporated by reference herein. Generally, these chimeric antibodies are constructed by preparing a genomic gene library from DNA extracted from pre-existing murine hybridomas (Nishimura et al., Cancer Research, 47:999 (1987)). The library is then screened for variable region genes from both heavy and light chains exhibiting the correct antibody fragment rearrangement patterns. Alternatively, cDNA libraries are prepared from RNA extracted from the hybridomas and screened, or the variable regions are obtained by polymerase chain reaction. The cloned variable region genes are then ligated into an expression vector containing cloned cassettes of the appropriate heavy or light chain human constant region gene. The chimeric genes can then be expressed in a cell line of choice, e.g., a murine myeloma line. Such chimeric antibodies have been used in human therapy.

Humanized antibodies are known in the art. Typically, “humanization” results in an antibody that is less immunogenic, with complete retention of the antigen-binding properties of the original molecule. In order to retain all the antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman variable domains onto human constant regions to generate a chimeric antibody (Morrison et al., Proc. Natl. Acad. Sci., USA 81:6801 (1984); Morrison and Oi, Adv. Immunol. 44:65 (1988) (which preserves the ligand-binding properties, but which also retains the immunogenicity of the nonhuman variable domains); (b) by grafting only the nonhuman CDRs onto human framework and constant regions with or without retention of critical framework residues (Jones et al. Nature, 321:522 (1986); Verhoeyen et al., Science 239:1539 (1988)); or (c) by transplanting the entire nonhuman variable domains (to preserve ligand-binding properties) but also “cloaking” them with a human-like surface through judicious replacement of exposed residues (to reduce antigenicity) (Padlan, Molec. Immunol. 28:489 (1991)).

Humanization by CDR grafting typically involves transplanting only the CDRs onto human fragment onto human framework and constant regions. Theoretically, this should substantially eliminate immunogenicity (except if allotypic or idiotypic differences exist). However, it has been reported that some framework residues of the original antibody also need to be preserved (Riechmann et al., Nature 332:323 (1988); Queen et al., Proc. Natl. Acad. Sci. USA 86:10,029 (1989)). The framework residues which need to be preserved can be identified by computer modeling. Alternatively, critical framework residues may potentially be identified by comparing known antibody combining site structures (Padlan, Molec. Immun. 31(3):169-217 (1994)). The compositions and methods of the disclosure also include partially humanized antibodies, in which the 6 CDRs of the heavy and light chains and a limited number of structural amino acids of the murine monoclonal antibody are grafted by recombinant technology to the CDR-depleted human IgG scaffold (Jones et al., Nature 321:522-525 (1986)).

Deimmunized antibodies are made by replacing immunogenic epitopes in the murine variable domains with benign amino acid sequences, resulting in a deimmunized variable domain. The deimmunized variable domains are linked genetically to human IgG constant domains to yield a deimmunized antibody (Biovation, Aberdeen, Scotland).

The anti-CD3 antibody can also be a single chain antibody. A single-chain antibody (scFV) can be engineered (see, for example, Colcher et al., Ann. N. Y. Acad. Sci. 880:263-80 (1999); and Reiter, Clin. Cancer Res. 2:245-52 (1996)). The single chain antibody can be dimerized or multimerized to generate multivalent antibodies having specificities for different epitopes of the same target CD3 protein. In some embodiments, the antibody is monovalent, e.g., as described in Abbs et al., Ther. Immunol. 1(6):325-31 (1994), incorporated herein by reference.

Exemplary anti-CD3 antibodies, comprise a heavy chain complementarity determining region 1 (CDRH1) comprising the amino acid sequence GYGMH (SEQ ID NO: 42), a heavy chain complementarity determining region 2 (CDRH2) comprising the amino acid sequence VIWYDGSKKYYVDSVKG (SEQ ID NO: 43), a heavy chain complementarity determining region 3 (CDRH3) comprising the amino acid sequence QMGYWHFDL (SEQ ID NO: 44), a light chain complementarity determining region 1 (CDRL1) comprising the amino acid sequence RASQSVSSYLA (SEQ ID NO: 45), a light chain complementarity determining region 2 (CDRL2) comprising the amino acid sequence DASNRAT (SEQ ID NO: 46), and a light chain complementarity determining region 3 (CDRL3) comprising the amino acid sequence QQRSNWPPLT (SEQ ID NO: 47).

In some embodiments, the anti-CD3 antibody comprises a variable heavy chain amino acid sequence comprising

QVQLVESGGGVVQPGRSLRLSCAASGFKFSGYGMHWVRQAPGKGLEWVAV
IWYDGSKKYYVDSVKGRFTISRDNSKNTLYLQMNSLRAEDTAVYYCARQM
GYWHFDLWGRGT

LVTVSS (SEQ ID NO: 48) and a variable light chain amino acid sequence comprising

EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPLTFG
GGTKVEIK(SEQ ID NO: 49).

Preferably, the anti-CD3 antibody comprises a heavy chain amino acid sequence comprising:

QVQLVESGGGVVQPGRSLRLSCAASGFKFSGYGMHWVRQAPGKGLEWVAV
IWYDGSKKYYVDSVKGRFTISRDNSKNTLYLQMNSLRAEDTAVYYCARQM
GYWHFDLWGRGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPEAEGGPSVFLFPPKPKD
TLMISRTPEVTCVWDVSHEDPEVKFNWYVDGVEVHNAKTKPREEQYNSTY
RVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
(SEQ ID NO: 50)

and a light chain amino acid sequence comprising:

EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPLTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC (SEQ ID NO: 51).

This anti-CD3 antibody is referred to herein as NI-0401, Foralumab, or 28F11-AE. (See e.g., Dean Y, Depis F, Kosco-Vilbois M. “Combination therapies in the context of anti-CD3 antibodies for the treatment of autoimmune diseases.” Swiss Med Wkly. (2012) (the contents of which are hereby incorporated by reference in its entirety).

In some embodiments, the anti-CD3 antibody is a fully human antibody or a humanized antibody. In some embodiments, the anti-CD3 antibody formulation includes a full length anti-CD3 antibody. In alternative embodiments, the anti-CD3 antibody formulation includes an antibody fragment that specifically binds CD3. In some embodiments, the anti-CD3 antibody formulation includes a combination of full-length anti-CD3 antibodies and antigen binding fragments that specifically bind CD3.

In some embodiments, the antibody or antigen-binding fragment thereof that binds CD3 is a monoclonal antibody, domain antibody, single chain, Fab fragment, a F(ab′)2 fragment, a scFv, a scAb, a dAb, a single domain heavy chain antibody, or a single domain light chain antibody. In some embodiments, such an antibody or antigen-binding fragment thereof that binds CD3 is a mouse, other rodent, chimeric, humanized or fully human monoclonal antibody.

Optionally, the anti-CD3 antibody or antigen binding fragment thereof used in the formulations of the disclosure includes at least one amino acid mutation. Typically, the mutation is in the constant region. The mutation results in an antibody that has an altered effector function. An effector function of an antibody is altered by altering, i.e., enhancing or reducing, the affinity of the antibody for an effector molecule such as an Fc receptor or a complement component. For example, the mutation results in an antibody that is capable of reducing cytokine release from a T-cell. For example, the mutation is in the heavy chain at amino acid residue 234, 235, 265, or 297 or combinations thereof. Preferably, the mutation results in an alanine residue at either position 234, 235, 265 or 297, or a glutamate residue at position 235, or a combination thereof.

Preferably, the anti-CD3 antibody provided herein contains one or more mutations that prevent heavy chain constant region-mediated release of one or more cytokine(s) in vivo.

In some embodiments, the anti-CD3 antibody or antigen binding fragment thereof used in the formulations of the disclosure is a fully human antibody. The fully human CD3 antibodies used herein include, for example, a L234A and L235E mutation in the Fc region, such that cytokine release upon exposure to the anti-CD3 antibody is significantly reduced or eliminated. The L234A and L235E mutation in the Fc region of the anti-CD3 antibodies provided herein reduces or eliminates cytokine release when the anti-CD3 antibodies are exposed to human leukocytes, whereas the mutations described below maintain significant cytokine release capacity. For example, a significant reduction in cytokine release is defined by comparing the release of cytokines upon exposure to the anti-CD3 antibody having an L234A and L235E mutation in the Fc region to level of cytokine release upon exposure to another anti-CD3 antibody having one or more of the mutations described below. Other mutations in the Fc region include, for example, L234A and L235A, L235E, N297A, D265A, or combinations thereof.

The term “cytokine” refers to all human cytokines known within the art that bind extracellular receptors expressed on the cell surface and thereby modulate cell function, including but not limited to IL-2, IFN-gamma, TNF-a, IL-4, IL-5, IL-6, IL-9, IL-10, and IL-13.

The anti-CD3 formulation comprises a unit dose of the anti-CD3 antibody in the range of: about 0.01 mg to about 25 mg; or 0.01 mg to about 10 mg. For example, the unit dose is about 0.01, 0.02, 0.03, 0.04, 0.50, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 5.0, 5.5, 6.0, 6.5, 7.0, 7.5, 8.0, 8.5, 9, 9.5, 10 mg or more. Preferably, the unit dose is 0.05 mg, 0.1 mg, 0.5 mg,1.0 mg, 2.5 mg, 5.0 mg or 10 mg.

The anti-CD3 antibody formulation includes one or more salts (a buffering salt), one or more polyols and one or more excipients. The formulations of the present disclosure may also contain buffering agents, or preservatives. The anti-CD3 antibody formulation is buffered in a solution at a pH in the range of about 4 to 8; in the range of about 4 to 7; in the range of about 4 to 6; in the range of about 5 to 6; or in the range of about 5.5 to 6.5. Preferably, the pH is 5.5.

Examples of salts include those prepared from the following acids: hydrochloric, hydrobromic, sulfuric, nitric, phosphoric, maleic, acetic, salicylic, citric, boric, formic, malonic, succinic, and the like. Such salts can also be prepared as alkaline metal or alkaline earth salts, such as sodium, potassium or calcium salts. Examples of buffering agents include phosphate, citrate, acetate, and 2-(N-morpholino)ethanesulfonic acid (MES).

The formulations of the present disclosure may include a buffer system. As used in this application, the terms “buffer” or “buffer system” is meant a compound that, usually in combination with at least one other compound, provides a buffering system in solution that exhibits buffering capacity, that is, the capacity to neutralize, within limits, either acids or bases (alkali) with relatively little or no change in the original pH.

Buffers include borate buffers, phosphate buffers, calcium buffers, and combinations and mixtures thereof. Borate buffers include, for example, boric acid and its salts, for example, sodium borate or potassium borate. Borate buffers also include compounds such as potassium tetraborate or potassium metaborate that produce borate acid or its salt in solutions.

A phosphate buffer system includes one or more monobasic phosphates, dibasic phosphates and the like. Particularly useful phosphate buffers are those selected from phosphate salts of alkali and/or alkaline earth metals. Examples of suitable phosphate buffers include one or more of sodium dibasic phosphate (Na2HPO4), sodium monobasic phosphate (NaH2PO4) and potassium monobasic phosphate (KH2PO4). The phosphate buffer components frequently are used in amounts from 0.01% or to 0.5% (w/v), calculated as phosphate ion.

Other known buffer compounds can optionally be added to the according to the CD3 formulations, for example, citrates, sodium bicarbonate, TRIS, and the like. Other ingredients in the solution, while having other functions, may also affect the buffer capacity. For example, EDTA, often used as a complexing agent, can have a noticeable effect on the buffer capacity of a solution.

Preferred salts for use in the formulation of the disclosure include sodium chloride, sodium acetate, sodium acetate trihydrate and sodium citrate.

The concentration of salt in the formulations according to the disclosure is between about 10 mM and 500 mM, between about 25 m and 250 mM, between about 25 nM and 150 mM.

The sodium acetate trihydrate is at a concentration in the range of about 10 mM to 100 mM. For example, the sodium acetate trihydrate is at about 10, 15, 20, 25, 30, 35, 40, 45, 50, 55, 60, 65, 70, 75, 80, 85, 90, 95 or 100 mM. Preferably, the sodium acetate trihydrate is at 25 mM.

The sodium chloride at a concentration in the range of about 50 mM to 500 mM. For example, the sodium chloride is at about 50, 55, 60, 65, 70, 75, 80, 85, 90, 95, 100. 125, 150, 175, 200, 225, 250, 275, 300, 325, 350, 375, 400, 425, 450, 475 or 500 mM. Preferably, the sodium chloride is at a concentration of about 125 mM.

The sodium citrate is at a concentration in the range of about 10 mM to 100 mM For example the sodium citrate is at about 10, 15, 20, 25, 30, 35, 40, 45, 50, 55, 60, 65, 70, 75, 80, 85, 90, 95 or 100 mM. Preferably, the sodium citrate is in the range of about 25 to 50 mM.

In some embodiments, the salt is sodium acetate trihydrate at a concentration in the range of about 25 mm to 100 mm and sodium chloride at a concentration in the range of about 150 mm to 500 mm.

Preferably, the formulation includes about 25 mM sodium acetate trihydrate and about 150 mM sodium chloride.

The formulation includes one or more polyols as a bulking agent and/or stabilizing excipients. Polyols include for example, trehalose, mannitol, maltose, lactose, sucrose, sorbitol, or glycerol. The polyols is at a concentration in the range of about 0.1% to 50% or 5% to 25%. For example, the polyol is at about 1, 2, 3, 4, 5, 10, 15, 20, 25, 30, 35, 40, 45 or 50%.

In some embodiments, the polyol is trehalose at a concentration in the range of about 1% to 50% or 5% to 25%. For example, the trehalose is at about 1, 2, 3, 4, 5, 10, 15, 20, 25, 30, 35, 40, 45 or 50%. Preferably the trehalose is at a concentration of about 10% or about 20%. Most preferably, the trehalose is at a concentration of about 20%.

In some embodiments, the polyol is sorbitol at a concentration in the range of about 1% to about 10%. In some embodiments, the polyol is glycerol at a concentration in the range of about 1% to about 10%.

In some embodiments, the polyol is mannitol at a concentration in the range of about 0.1% to about 10%. In some embodiments, the polyol is maltose at a concentration in the range of about 1% to about 10%.

The formulation includes one or more excipients and/ or surfactants to suppress or otherwise reduce antibody aggregation. Suitable excipients to reduce antibody aggregation include, by way of non-limiting example, a surfactant such as, by way of non-limiting example, Polysorbate 20 or Polysorbate 80. In some embodiments, the Polysorbate 20 or Polysorbate 80 is present at a concentration in the range of about 0.01 to 1 % or about 0.01 to 0.05%. For example the Polysorbate 20 or Polysorbate 80 is at a concentration of about 0.01. 0.02, 0.03, 0.04, 0.05, 0.06, 0.07. 0.08, 0.09, 0.1, 0.2, 0.3. 0.4, 0.5, 0.6, 0.7, 0.8. 0.9, or 1.0 %.

Preferably the surfactant is Polysorbate 80 at a concentration in the range of about 0.01 to 0.05%. More preferably, the Polysorbate 80 is at 0.02%.

The formulation includes one or more excipients to reduce antibody oxidation. Suitable excipients to reduce antibody oxidation include, by way of non-limiting example, antioxidants. Antioxidants include for example, methionine, D-arginine, BHT or ascorbic acid. The antioxidant is present at a concentration in the range of about 0.01 % to 1% ; 0.1% to 1%; or 0.1% to 0.5%. In some embodiments, the antioxidant is methionine. In some embodiments, the methionine is present at a concentration in the range of about 0.01 % to 1% ; 0.1% to 1%; or 0.1% to 0.5%. For example, the methionine is present at a concentration of about 0.01. 0.02, 0.03, 0.04, 0.05, 0.06, 0.07. 0.08, 0.09, 0.1, 0.2, 0.3. 0.4, 0.5, 0.6, 0.7, 0.8. 0.9, or 1.0 %. Preferably, the methionine is at about 0.1%.

The formulation includes one or more chelating agents, such as for example ethylenediaminetetraacetic acid (EDTA). The chelating agent is at a concentration in the range of 0.01 % to 1% ; 0.1% to 1%; or 0.1% to 0.5%. For example, the chelating agent is present at a concentration of about 0.01. 0.02, 0.03, 0.04, 0.05, 0.06, 0.07. 0.08, 0.09, 0.1, 0.2, 0.3. 0.4, 0.5, 0.6, 0.7, 0.8. 0.9, or 1.0 %. Preferably, the chelating agent is EDTA at a concentration of about 0.1%.

In some embodiments, the formulation includes one or more excipients to increase stability. In some embodiments, the excipient to increase stability is human serum albumin. In some embodiments, the human serum albumin is present in the range of about 1 mg to about 5 mg.

In some embodiments, the formulation includes magnesium stearate (Mg stearate), an amino acid, or both mg-stearate and an amino acid. Suitable amino acids include for example, leucine, arginine, histidine, or combinations thereof.

In some embodiments the one or more additional excipients is low moisture microcrystalline cellulose, such as Avicel, polyethylene glycols (PEG), or a starch.

Further examples of pharmaceutically acceptable carriers and excipients useful for the formulations of the present disclosure include, but are not limited to binders, fillers, disintegrants, lubricants, anti-microbial agents, antioxidant, and coating agents such as: BINDERS: corn starch, potato starch, other starches, gelatin, natural and synthetic gums such as acacia, xanthan, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone (e.g., povidone, crospovidone, copovidone, etc), methyl cellulose, Methocel, pre-gelatinized starch (e.g., STARCH 1500® and STARCH 1500 LM®, sold by Colorcon, Ltd.), hydroxypropyl methyl cellulose, microcrystalline cellulose (FMC Corporation, Marcus Hook, PA, USA), Emdex, Plasdone, or mixtures thereof, FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, dextrose, fructose, honey, lactose anhydrate, lactose monohydrate, lactose and aspartame, lactose and cellulose, lactose and microcrystalline cellulose, maltodextrin, maltose, mannitol, microcrystalline cellulose &amp; guar gum, molasses, sucrose,or mixtures thereof, DISINTEGRANTS: agar-agar, alginic acid, calcium carbonate, microcrystalline cellulose, croscarmellose sodium, crospovidone, polacrilin potassium, sodium starch glycolate, (such as Explotab), potato or tapioca starch, other starches, pre-gelatinized starch, clays, other algins, other celluloses, gums (like gellan), low-substituted hydroxypropyl cellulose, ployplasdone, or mixtures thereof, LUBRICANTS: calcium stearate, magnesium stearate, mineral oil, light mineral oil, glycerin, sorbitol, mannitol, polyethylene glycol, other glycols, compritol, stearic acid, sodium lauryl sulfate, sodium stearyl fumarate, (such as Pruv), vegetable based fatty acids lubricant, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, MD USA), a coagulated aerosol of synthetic silica (Deaussa Co., Piano, TX USA), a pyrogenic silicon dioxide (CAB-O-SIL, Cabot Co., Boston, MA USA), or mixtures thereof, ANTI-CAKING AGENTS: calcium silicate, magnesium silicate, silicon dioxide, colloidal silicon dioxide, talc, or mixtures thereof, ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, or mixtures thereof, ANTOXIDANTS: ascorbic acid, BHA, BHT, EDTA, or mixture thereof, and COATING AGENTS: sodium carboxymethyl cellulose, cellulose acetate phthalate, ethylcellulose, gelatin, pharmaceutical glaze, hydroxypropyl cellulose, hydroxypropyl methylcellulose (hypromellose), hydroxypropyl methyl cellulose phthalate, methylcellulose, polyethylene glycol, polyvinyl acetate phthalate, shellac, sucrose, titanium dioxide, carnauba wax, microcrystalline wax, gellan gum, maltodextrin, methacrylates, microcrystalline cellulose and carrageenan or mixtures thereof.

The formulation can also include other excipients and categories thereof including but not limited to Pluronic®, Poloxamers (such as Lutrol® and Poloxamer 188), ascorbic acid, glutathione, protease inhibitors (e.g. soybean trypsin inhibitor, organic acids), pH lowering agents, creams and lotions (like maltodextrin and carrageenans); materials for chewable tablets (like dextrose, fructose, lactose monohydrate, lactose and aspartame, lactose and cellulose, maltodextrin, maltose, mannitol, microcrystalline cellulose and guar gum, sorbitol crystalline); parenterals (like mannitol and povidone); plasticizers (like dibutyl sebacate, plasticizers for coatings, polyvinylacetate phthalate); powder lubricants (like glyceryl behenate); soft gelatin capsules (like sorbitol special solution); spheres for coating (like sugar spheres); spheronization agents (like glyceryl behenate and microcrystalline cellulose); suspending/gelling agents (like carrageenan, gellan gum, mannitol, microcrystalline cellulose, povidone, sodium starch glycolate, xanthan gum); sweeteners (like aspartame, aspartame and lactose, dextrose, fructose, honey, maltodextrin, maltose, mannitol, molasses, sorbitol crystalline, sorbitol special solution, sucrose); wet granulation agents (like calcium carbonate, lactose anhydrous, lactose monohydrate, maltodextrin, mannitol, microcrystalline cellulose, povidone, starch), caramel, carboxymethylcellulose sodium, cherry cream flavor and cherry flavor, citric acid anhydrous, citric acid, confectioner’s sugar, D&C Red No. 33, D&C Yellow #10 Aluminum Lake, disodium edetate, ethyl alcohol 15%, FD&C Yellow No. 6 aluminum lake, FD&C Blue # 1 Aluminum Lake, FD&C Blue No. 1, FD&C blue no. 2 aluminum lake, FD&C Green No.3, FD&C Red No. 40, FD&C Yellow No. 6 Aluminum Lake, FD&C Yellow No. 6, FD&C Yellow No.10, glycerol palmitostearate, glyceryl monostearate, indigo carmine, lecithin, manitol, methyl and propyl parabens, mono ammonium glycyrrhizinate, natural and artificial orange flavor, pharmaceutical glaze, poloxamer 188, Polydextrose, polysorbate 20, polysorbate 80, polyvidone, pregelatinized corn starch, pregelatinized starch, red iron oxide, saccharin sodium, sodium carboxymethyl ether, sodium chloride, sodium citrate, sodium phosphate, strawberry flavor, synthetic black iron oxide, synthetic red iron oxide, titanium dioxide, and white wax.

The CD3 antibodies formulated for enteral, parenteral, or nasal administration. For example, the CD3 antibodies are formulated for nasal, oral, inhalation, subcutaneous or intravenous administration.

For enteral administration, i.e., oral, the formulations may be a capsule or a tablet. Parental administration includes intravenous, subcutaneous, intramuscular, and intra-articular administration and may be a liquid or lyophilized powder in a sealed vial or other container. Preferred oral dose ranges is 0.1 mg to 5 mg daily. For example, a dose 0.1 mg, 0.2 mg , 0.3 mg, 0.4 mg, 0.5 mg, 0.6 mg, 0.7 mg, 0.8 mg, 0.9 mg, 1.0 mg, 1.5 mg, 2.0 mg, 2.5 mg, 3.0 mg, 3.5 mg , 4.0 mg , or 5.0 mg is administered daily. Administration of the dose is once daily or twice daily.

For nasal administration, the formulations may be an aerosol in a sealed vial or other suitable container. Preferred nasal dose ranges is 0.05 mg to 1 mg daily. For example, a dose 0.05 mg, 0.06 mg, 0.07 mg, 0.08 mg, 0.09 mg, 0.1 mg, 0.2 mg , 0.3 mg, 0.4 mg, 0.5 mg, 0.6 mg, 0.7 mg, 0.8 mg, 0.9 mg, or 1.0 mg, is administered daily. The does is equally split between each nostril. Administration of the dose is once daily or twice daily.

In some embodiments, the anti-CD3 antibody formulation is a subcutaneous formulation. In some embodiments, the subcutaneous anti-CD3 antibody formulation is housed in a sealed vial or other container. Preferred subcutaneous dose ranges is 0.2 mg to 5 mg daily. For example, a dose 0.2 mg, 0.3 mg, 0.4 mg, 0.5 mg, 0.6 mg, 0.7 mg, 0.8 mg, 0.9 mg, 1.0 mg, 1.5 mg, 2.0 mg , 2.5 mg , 3.0 mg , 3.5 mg , 4.0 mg , or 5.0 mg is administered daily. Administration of the dose is once daily or twice daily. A preferred formulation for subcutaneous administration is a preferred dosage of anti-CD3 antibody in 25 mM sodium acetate buffer, 125 mM sodium chloride with 0.02% polysorbate 80, at pH 5.5.

In some embodiments, the anti-CD3 antibody formulation is inhalation formulation. For inhalation administration, the formulations may be an aerosol in a sealed vial or other suitable container. Administration by inhalation may be in the form of an inhaler or a nebulizer. The nebulizer and/or inhaler is handheld. Optionally, the nebulizer and/or inhaler can be of different sizes to fit children and/or adults.

Preferred inhalation dose ranges is 0.1 mg to 5 mg daily. For example, a dose 0.1 mg, 0.2 mg, 0.3 mg, 0.4 mg, 0.5 mg, 0.6 mg, 0.7 mg, 0.8 mg, 0.9 mg, 1.0 mg, 1.5 mg, 2.0 mg, 2.5 mg , 3.0 mg , 3.5 mg , 4.0 mg , or 5.0 mg is administered daily. Administration of the dose is once daily or twice daily.

Particles of a particle formulation have diameters of between about 1 mm to about 5 mm, e.g., less than 5 mm in diameter, less than 4 mm in diameter, less than 3 mm in diameter, less than 2 mm in diameter, and about 1 mm in diameter.

Particles of a particle formulation comprising an anti-CD3 antibody or antigen-binding fragment thereof have average diameters of between about 0.1 mm to about 50 mm. Particles of a particle formulation comprising an anti-CD3 antibody or antigen-binding fragment thereof have average diameters of between about 1 mm to about 10 mm, e.g., less than 10 mm in average diameter, less than 9 mm in average diameter, less than 8 mm in average diameter, less than 7 mm in average diameter, less than 6 mm in average diameter, less than 5 mm in average diameter, less than 4 mm in average diameter, less than 3 mm in average diameter, and about 2 mm in average diameter. In some embodiments, particles have average diameters of between about 2 mm and 5 mm. In some embodiments, the particles have an average diameter between 2 mm and 5 mm, where each particle is less than about 50 mm in diameter.

In some embodiments the CD3 antibody is an extended and controlled release formulation. Methods of producing extended and controlled release formulation are known in the art and includes for example the use or macroporous beads.

In some embodiments, the anti-CD3 antibody formulation includes a full length anti-CD3 antibody. In some embodiments, the anti-CD3 antibody formulation includes an antibody fragment that specifically binds CD3. In some embodiments, the anti-CD3 antibody formulation includes a combination of full-length anti-CD3 antibodies and antigen binding fragments that specifically bind CD3.

### IL-6/IL-6 Receptor Antibodies

Exemplary IL-6/IL-6 receptor (IL-6R) antibodies useful in the compositions and methods of the disclosure include, for example, Actemra® (tocilizumab), or Kevzera® (sarilumab) and anti-IL-6 mAbs.

Other, IL-6R antibodies include the 39B9 VL1 antibody, the 39B9 VL5 antibody, the 12A antibody, and the 5C antibody described in WO/2009/140348, the contents of which are hereby incorporated by reference in its entirety. These antibodies show specificity for human IL-6R and/or both IL-6R and IL-6Rc and they have been shown to inhibit the functional activity of IL-6Rc (i.e., binding to gp130 to induce the signaling cascade) in vitro.

In some embodiments, the anti-6Rc antibody comprises a light chain and a heavy chain sequence. In some embodiments, the anti-6R antibody light chain sequence is SEQ ID NO: 53. In some embodiments, the anti-6Rc antibody heavy chain sequence is SEQ ID NO: 52. In some embodiments, the anti-6Rc antibody comprises SEQ ID NO: 53 and SEQ ID NO: 52.

The 39B9 VL1 and 39B9 VL5 antibodies share a common heavy chain variable region (SEQ ID NO:2) encoded by the nucleic acid sequence shown in SEQ ID NO: 1. The 39B9 VL1 antibody includes a light chain variable region (SEQ ID NO: 4) encoded by the nucleic acid sequence shown in SEQ ID NO: 3. The 39B9 VL5 antibody includes a light chain variable region (SEQ ID NO: 6) encoded by the nucleic acid sequence shown in SEQ ID NO: 5. The 12A antibody includes a heavy chain variable region (SEQ ID NO:8) encoded by the nucleic acid sequence shown in SEQ ID NO: 7. The 12A antibody includes a light chain variable region (SEQ ID NO: 10) encoded by the nucleic acid sequence shown in SEQ ID NO: 9. The 5C antibody includes a heavy chain variable region (SEQ ID NO: 12) encoded by the nucleic acid sequence shown in SEQ ID NO: 11. The 5C antibody includes a light chain variable region (SEQ ID NO: 14) encoded by the nucleic acid sequence shown in SEQ ID NO: 13.

TABLE 1

Illustrative IL-6/IL-6R Antibody Sequences

| Name | SEQ ID NO. | Sequence |
|---|---|---|
| 39B9 VL1-VH nucleic acid sequence | 1 | CAGGTGCAGCTGGTGCAGTCTGGGGCTGAGGTGAAGAAGCCTGGGTCCTCGGTGAAGGTCTCCTGCAAGGCTTCTGGAGGCACCTTCAGCAGCTATGCTATCAGCTGGGTGCGCCAGGCCCCTGGACAAGGGCTTGAGTGGATGGGAGGGATCATCCCTCTCTTTGATACAACAAAGTACGCACAGCAGTTCCAGGGCAGAGTCACGATTACCGCGGACGAATCCACGAGCACAGCCTACATGGAGCTGAGCAGCCTGAGATCTGAGGACACGGCCGTATTTTACTGTGCGAGAGATCGGGATATTTTGACTGATTATTATCCCATGGGCGGTATGGACGTCTGGGGCCAAGGGACCACGGTCACCGTCTCCTCA |
| 39B9 VL1-VH amino acid sequence | 2 | QVQLVQSGAEVKKPGSSVKVSCKASGGTFSSYAISWVRQAPGQGLEWMGGIIPLFDTTKYAQQFQGRVTITADESTSTAYMELSSLRSEDTAVFYCARDRDILTDYYPMGGMDVWGQGTTVTVSS |
| 39B9 VL1-VL nucleic acid sequence | 3 | GCCATCCAGTTGACCCAGTCTCCATCCTCCCTGTCTGCATCTGTAGGAGACAGAGTCACCATCACTTGCCGGGCAAGTCAGGGCATTAGCAGTGTTTTAGCCTGGTATCAGCAGAAACCAGGGAAAGCTCCTAAGCTCCTGATCTATGATGCCTCCAGTTTGGAAAGTGGGGTCCCATCAAGGTTCAGCGGCAGTGGATCTGGGACAGATTTCACTCTCACCATCAGCAGCCTGCAGCCTGAAGATTTTGCAACTTATTACTGTCAACAGTCTAATAGTTACCCGCTCACTTTCGGCGGAGGGACCAAGGTGGAGATCAAACGT |
| 39B9 VL1-VL amino acid sequence | 4 | AIQLTQSPSSLSASVGDRVTITCRASQGISSVLAWYQQKPGKAPKLLIYDASSLESGVPSRFSGSGSGTDFTLTISSLQPEDFATYYCQQSNSYPLTFGGGTKVEIKR |
| 39B9 VL5-VL nucleic acid sequence | 5 | GACATCCTGATGACCCAGTCTCCATCCTCCCTGTCTGCATCTGTAGGAGACAGAGTCACCATCACTTGTCGGGCGAGTCAGGATATTAGCAGCTGGTTAGCCTGGTATCAGCAGAAACCAGGGAAAGCTCCTAAGCTCCTGATCTATGATGCCTCCAGTTTGGAAAGTGGGGTCCCATCAAGGTTCAGCGGCAGTGGATCTGGGACAGATTTCACTCTCACCATCAGCAGCCTGCAGCCTGAAGATTTTGCAACTTATTACTGTCAACAGTCTAATAGTTACCCGCTCACTTTCGGCGGAGGGACCAAGGTGGAGATCAAACGA |
| 39B9 VL5-VL amino acid sequence | 6 | DILMTQSPSSLSASVGDRVTITCRASQDISSWLAWYQQKPGKAPKLLIYDASSLESGVPSRFSGSGSGTDFTLTISSLQPEDFATYYCQQSNSYPLTFGGGTKVEIKR |
| 12A VH nucleic acid sequence | 7 | CAGGTGCAGCTGGTGGAGTCTTGGGGAGGCGTGGTCCAGCCTGGGAGGTCCCTGAGACTCTCCTGTGCAGCGTCTGGATTCACCTTCAGTAACTATGACATGTACTGGGTCCGCCAGGCTCCAGGCAAGGGGCTGGAGTGGGTGGCAGTTATATTAGATGATGGAAATAATAATTACTACGCAGACTCCGTGAAGGGCCGATTCACCATCTCCAGAGACAATTCCAAGAAAAAGGTGTATCTGCAAATGAATAGCCTGAGAGCTGAGGACACGGCTGTGTATTACTGTGTGAGAGCGTCCCCTAACTGGGGTCTTCTTGACTTCTGGGGCCAGGGAACCCTGGTCACCGTCTCGAGT |
| 12A VH amino acid sequence | 8 | QVQLVESWGGWQPGRSLRLSCAASGFTFSNYDMYWVRQAPGKGLEWVAVILDDGNNNYYADSVKGRFTISRDNSKKKVYLQMNSLRAEDTAVYYCVRASPNWGLLDFWGQGTLVTVSS |
| 12A VL nucleic acid sequence | 9 | GAAATTGTGTTGACACAGTCTCCATCCTCACTGTCTGCATCTGTAGGAGACAGAGTCACCATCACTTGTCGGGCGAGTCAGGGTATTAGCAGCTGGTTAGCCTGGTATCAGCAGAAACCAGGGAAAGCTCCTAAGCTCCTGATCTATGATGCCTCCAGTTTGGAAAGTGGGGTCCCATCAAGGTTCAGCGGCAGTGGATCTGGGACAGATTTCACTCTCACCATCAGCAGCCTGCAGCCTGAAGATTTTGCAACTTATTACTGTCAACAGTTTAATAGTTACCCGATCACCTTCGGCCAAGGGACACGACTGGAGATTAAACGT |
| 12A VL amino acid sequence | 10 | EIVLTQSPSSLSASVGDRVTITCRASQGISSWLAWYQQKPGKAPKLLIYDASSLESGVPSRFSGSGSGTDFTLTISSLQPEDFATYYCQQFNSYPITFGQGTRLEIKR |
| 5C VH nucleic acid sequence | 11 | CAGGTGCAGCTGGTGCAGTCTGGGGGAGGCGTGGTCCAGCCTGGGAGGTCCCTGAGACTCTCCTGTGCAGCCTCTGGATTCATCTTCAGTAGCTATGACATGTACTGGGTCCGCCAGGCTCCAGGCAAGGGGCTGGAGTGGGTGGCAGTTATATTATATGATGGAAATAATAAATACTACGCAGACTCCGTGAAGGGCCGATTCACCATCTCCAGAGACAATTCCAAGAACACGGTGTATCTGCAAATGAACAGCCTGAGAGCTGAGGACACGGCTGTGTATTACTGTGTGAGAGCGTCCCCTAACTGGGGTCTTTTTGACTTCTGGGGCCAGGGAACCCTGGTCACCGTCTCGAGT |
| 5C VH amino acid sequence | 12 | QVQLVQSGGGVVQPGRSLRLSCAASGFIFSSYDMYWVRQAPGKGLEWVAVILYDGNNKYYADSVKGRFTISRDNSKNTVYLQMNSLRAEDTAVYYCVRASPNWGLFDFWGQGTLVTVSS |
| 5C VL nucleic acid sequence | 13 | GACATCCAGATGACCCAGTCTCCATCCTCCCTGTCTGCATCTGTAGGAGACAGAGTCACCATCACTTGCCGGGCAAGTCAGGGCATTAGCAGTGATTTAGCCTGGTATCAGCAGAAACCAGGGAAAGCTCCTAAGCTCCTGATGTATGATGCCTCCAGTTTGGAAAGTGGGGTCCCATCAAGGTTCAGCGGCAGTGGATCTGGGACAGATTTCACTCTCACCATCAGCAGCCTGCAGCCTGAAGATTTTGCAACTTATTACTGTCAACAGTTTAATAGTTACCCGATCACCTTCGGCCAAGGGACACGACTGGAGATTAAACGT |
| 5C VL amino acid sequence | 14 | DIQMTQSPSSLSASVGDRVTITCRASQGISSDLAWYQQKPGKAPKLLMYDASSLESGVPSRFSGSGSGTDFTLTISSLQPEDFATYYCQQFNSYPITFGQGTRLEIKR |

huIL-6R antibodies of the disclosure additionally comprise, for example, the heavy chain complementarity determining regions (VH CDRs) shown below in Table 2, the light chain complementarity determining regions (VL CDRs) shown in Table 3, and combinations thereof.

TABLE 2

VH CDR sequences from antibody clones that bind and neutralize IL-6R biological activity

| | | | |
|---|---|---|---|
| Clone Name | VH CDR1 | VH CDR2 | VH CDR3 |
| 39B9 | SYAIS (SEQ ID NO: 15) | GIIPLFDTTKYAQQFQG (SEQ ID NO: 16) | CARDRDILTDYYPMGGMDV (SEQ ID NO: 17) |
| 12A | NYDMY (SEQ ID NO: 18) | VILDDGNNNYYADSVKG (SEQ ID NO: 19) | CVRASPNWGLLDF (SEQ ID NO: 20) |
| 5C | SYDMY | VILYDGNNKYYADSVKG | CVRASPNWGLFDF |
| | (SEQ ID NO: 21) | (SEQ ID NO: 22) | (SEQ ID NO: 23) |

TABLE 3

VL CDR sequences from antibody clones that bind and neutralize IL-6Rc

| | | | |
|---|---|---|---|
| Clone Name | VL CDR1 | VL CDR2 | VL CDR3 |
| 39B9 VL1 | RASQGISSVLA (SEQ ID NO: 24) | DASSLES (SEQ ID NO: 25) | QQSNSYPLT (SEQ ID NO: 26) |
| 39B9 VL5 | RASQDISSWLA (SEQ ID NO: 27) | DASSLES (SEQ ID NO: 25) | QQSNSYPLT (SEQ ID NO: 26) |
| 12A | RASQGISSWLA (SEQ ID NO: 28) | DASSLES (SEQ ID NO: 25) | QQSNSYPIT (SEQ ID NO: 29) |
| 5C | RASQGISSVDA (SEQ ID NO: 30) | DASSLES (SEQ ID NO: 25) | QQSNSYPIT (SEQ ID NO: 29) |

The huIL-6R antibodies of the disclosure serve to modulate, block, inhibit, reduce, antagonize, neutralize or otherwise interfere with the functional activity of IL-6Rc. Functional activities of IL-6Rc include for example, intracellular signaling via activation of the JAK/STAT pathway and activation of the MAPK cascade, acute phase protein production, antibody production and cellular differentiation and/or proliferation. For example, the huIL-6R antibodies completely or partially inhibit IL-6Rc functional activity by partially or completely modulating, blocking, inhibiting, reducing antagonizing, neutralizing, or otherwise interfering with the binding of IL-6Rc to the signal-transducing receptor component gp130.

The huIL-6R antibodies are considered to completely modulate, block, inhibit, reduce, antagonize, neutralize or otherwise interfere with IL-6Rc functional activity when the level of IL-6Rc functional activity in the presence of the huIL-6R antibody is decreased by at least 95%, e.g., by 96%, 97%, 98%, 99% or 100% as compared to the level of IL-6Rc functional activity in the absence of binding with a huIL-6R antibody described herein. The huIL-6R antibodies are considered to partially modulate, block, inhibit, reduce, antagonize, neutralize or otherwise interfere with IL-6Rc functional activity when the level of IL-6Rc activity in the presence of the huIL-6R antibody is decreased by less than 95%, e.g., 10%, 20%, 25%, 30%, 40%, 50%, 60%, 75%, 80%, 85% or 90% as compared to the level of IL-6Rc activity in the absence of binding with a huIL-6R antibody described herein.

### Variants of huIL-6R Antibodies

Variants of the huIL-6R antibodies are made using any of a variety of art-recognized techniques. For example, variant huIL-6R antibodies include antibodies having one or more amino acid modifications, such as, for example, an amino acid substitution, at position within the antibody sequence.

Preferred locations for amino acid substitutions are shown as bold, underlined residues below in Table 4. The amino acid residues in bold/underline can be replaced with any amino acid residue. In preferred embodiments, the amino acid residues in bold/underline are replaced with the amino acid residues shown below in Table 4. In these embodiments, the antibody comprises (i) the consensus amino acid sequence QQSXSYPLT (SEQ ID NO: 31) in the light chain complementarity determining region 3 (CDR3), where X is N or Q; (ii) the consensus amino acid sequence GIIPX1FX2TTKYAQX3FQG (SEQ ID NO: 32) in the heavy chain complementarity determining region 2 (CDR2), where X1 is L or A, X2 is D or E, and X3 is Q or K; (iii) the consensus amino acid sequence DRDILTDYYPXGGMDV (SEQ ID NO: 33) in the heavy chain complementarity determining region 3 (CDR3), where X is M or L; and (iv) the consensus amino acid sequence TAVXYCAR (SEQ ID NO: 34) in the framework region 3 (FRW3), where X is F or Y.

The NI-1201-wild type (NI-1201-WT) antibody listed in Table 4 comprises the amino acid sequence QQSNSYPLT (SEQ ID NO: 26) in the light chain CDR3 region, the amino acid sequence GIIPLFDTTKYAQQFQG (SEQ ID NO: 16) in the heavy chain CDR2 region, the amino acid sequence DRDILTDYYPMGGMDV (SEQ ID NO: 35) in the heavy chain CDR3 region, and the amino acid sequence TAVFYCAR (SEQ ID NO: 36) in the FRW3 region.

The NI-1201-A antibody listed in Table 4 comprises the amino acid sequence QQSNSYPLT (SEQ ID NO: 26) in the light chain CDR3 region, the amino acid sequence GIIPLFDTTKYAQKFQG (SEQ ID NO: 37) in the heavy chain CDR2 region, the amino acid sequence DRDILTDYYPMGGMDV (SEQ ID NO: 35) in the heavy chain CDR3 region, and the amino acid sequence TAVYYCAR (SEQ ID NO: 38) in the FRW3 region.

The NI-1201-B antibody listed in Table 4 comprises the amino acid sequence QQSNSYPLT (SEQ ID NO: 26) in the light chain CDR3 region, the amino acid sequence GIIPLFDTTKYAQKFQG (SEQ ID NO: 37) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

The NI-1201-C antibody listed in Table 4 comprises the amino acid sequence QQSNSYPLT (SEQ ID NO: 26) in the light chain CDR3 region, the amino acid sequence GIIPAFETTKYAQKFQG (SEQ ID NO: 40) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

The NI-1201-D antibody listed in Table 4 comprises the amino acid sequence QQSQSYPLT (SEQ ID NO: 41) in the light chain CDR3 region, the amino acid sequence GIIPAFETTKYAQKFQG (SEQ ID NO: 40) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

The NI-1201-E antibody listed in Table 4 comprises the amino acid sequence QQSQSYPLT (SEQ ID NO: 41) in the light chain CDR3 region, the amino acid sequence GIIPLFDTTKYAQKFQG (SEQ ID NO: 37) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

The NI-1201-F antibody listed in Table 4 comprises the amino acid sequence QQSNSYPLT (SEQ ID NO: 26) in the light chain CDR3 region, the amino acid sequence GIIPAFDTTKYAQKFQG (SEQ ID NO: 42) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

The NI-1201-G antibody listed in Table 4 comprises the amino acid sequence QQSQSYPLT (SEQ ID NO: 41) in the light chain CDR3 region, the amino acid sequence GIIPAFDTTKYAQKFQG (SEQ ID NO: 42) in the heavy chain CDR2 region, the amino acid sequence DRDILTDYYPLGGMDV (SEQ ID NO: 39) in the heavy chain CDR3 region, and the amino acid sequence TAVYYCAR (SEQ ID NO: 38) in the FRW3 region.

TABLE 4

NI-1201 Leads

| | | | | |
|---|---|---|---|---|
| | Light Chain CDR3 | Heavy Chain CDR2 | Heavy Chain CDR3 | FRW 3 |
| NI-1201-WT | QQSNSYPLT (SEQ ID NO: 26) | GIIPLFDTTKYAQQFQG (SEQ ID NO: 16) | DRDILTDYYPMGGMDV (SEQ ID NO: 35) | TAVFYCAR (SEQ ID NO: 36) |
| NI-1201-A | QQSNSYPLT (SEQ ID NO: 26) | GIIPLFDTTKYAQKFQG (SEQ ID NO: 37) | DRDILTDYYPMGGMDV(SEQ ID NO: 35) | TAVYYCAR (SEQ ID NO: 38) |
| NI-1201-B | QQSNSYPLT (SEQ ID NO: 26) | GIIPLFDTTKYAQKFQG (SEQ ID NO: 37) | DRDILTDYYPLGGMDV (SEQ ID NO: 39) | TAVYYCAR (SEQ ID NO: 38) |
| NI-1201-C | QQSNSYPLT (SEQ ID NO: 26) | GIIPAFETTKYAQKFQG (SEQ ID NO: 40) | DRDILTDYYPLGGMDV (SEQ ID NO: 39) | TAVYYCAR (SEQ ID NO: 38) |
| NI-1201-D | QQSQSYPLT (SEQ ID NO: 41) | GIIPAFETTKYAQKFQG (SEQ ID NO: 40) | DRDILTDYYPLGGMDV (SEQ ID NO: 39) | TAVYYCAR (SEQ ID NO: 38) |
| NI-1201-E | QQSQSYPLT (SEQ ID NO: 41) | GIIPLFDTTKYAQKFQG (SEQ ID NO: 37) | DRDILTDYYPLGGMDV (SEQ ID NO: 39) | TAVYYCAR (SEQ ID NO: 38) |
| NI-1201-F | QQSNSYPLT (SEQ ID NO: 26) | GIIPAFDTTKYAQKFQG (SEQ ID NO: 42) | DRDILTDYYPLGGMDV (SEQ ID NO: 39) | TAVYYCAR (SEQ ID NO: 38) |
| NI-1201-G | QQSQSYPLT (SEQ ID NO: 41) | GIIPAFDTTKYAQKFQG (SEQ ID NO: 42) | DRDILTDYYPLGGMDV (SEQ ID NO: 39) | TAVYYCAR (SEQ ID NO: 38) |

### CD28 Antibodies

Antibodies specific for the CD28 and antigen binding fragments thereof are referred to herein as an anti- CD28 antibody, and the compositions are referred to herein as an “anti- IL6/IL6 receptor antibody compositions.” Any anti- CD28 receptor antibody known in the art is suitable for use in the present disclosure. The anti- CD28 receptor antibody is a monoclonal antibody.

CD28 (Cluster of Differentiation 28) is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular).

CD28 is the receptor for CD80 (B7.1) and CD86 (B7.2) proteins. When activated by Toll-like receptor ligands, the CD80 expression is upregulated in antigen-presenting cells (APCs). The CD86 expression on antigen-presenting cells is constitutive (expression is independent of environmental factors).

### Phosphatidylinositol 3-Kinase (PI3K) Inhibitors

PI3K inhibitors are members of a unique and conserved family of intracellular lipid kinases that phosphorylate the 3′-OH group on phosphatidylinositols or phosphoinositides. PI3K inhibitors are key signaling enzymes that relay signals from cell surface receptors to downstream effectors. The PI3K family comprises 15 kinases with distinct substrate specificities, expression patterns, and modes of regulation. The class I PI3K inhibitors are typically activated by tyrosine kinases or G-protein coupled receptors to generate PIP3, which engages downstream effectors such as those in the Akt/PDK1 pathway, mTOR, the Tec family kinases, and the Rho family GTPases.

The PI3K signaling pathway is known to be one of the most highly mutated in human cancers. PI3K signaling is also a key factor in disease states including hematologic malignancies, non-Hodgkin’s lymphoma (such as diffuse large B-cell lymphoma), allergic contact dermatitis, rheumatoid arthritis, osteoarthritis, inflammatory bowel diseases, chronic obstructive pulmonary disorder, psoriasis, multiple sclerosis, asthma, disorders related to diabetic complications, and inflammatory complications of the cardiovascular system such as acute coronary syndrome. The PI3K-delta and PI3K gamma isoforms are preferentially expressed in normal and malignant leukocytes.

Downstream mediators of the PI3K signal transduction pathway include Akt and mammalian target of rapamycin (mTOR). One important function of Akt is to augment the activity of mTOR, through phosphorylation of TSC2 and other mechanisms. mTOR is a serine-threonine kinase related to the lipid kinases of the PI3K family and has been implicated in a wide range of biological processes including cell growth, cell proliferation, cell motility and survival.

The PI3K inhibitor can be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the disclosure.

The PI3K inhibitor is an antibody or fragment thereof that inhibits PI3K activity.

PI3K inhibitors are well known in the art, including those that are PI3K-delta inhibitors, PI3K gamma. inhibitors and those that are PI3K.delta/gamma inhibitors.

Exemplary PI3K inhibitors include for example, Wortmannin, LY294002, hibiscone C, Idelalisib, Copanlisib, Duvelisib, Alpelisib, Taselisib, Perifosine, Idelalisib, Buparlisib, Umbralisib, PX-866, Dactolisib, CUDC-907,Voxtalisib, CUDC-907, ME-401, IPI-549, SF1126 , RP6530, INK1117, pictilisib, XL147, Palomid 529, GSK1059615, ZSTK474, PWT33597, IC87114, TG100-115, RP6503, PI-103, GNE-477, and AEZS-136.

### Protein Kinase B (AKT) Inhibitors

Protein kinase B (PKB), also known as Akt, is a serine/threonine-specific protein kinase that plays a key role in multiple cellular processes such as glucose metabolism, apoptosis, cell proliferation, transcription, and cell migration.

Akt1 is involved in cellular survival pathways, by inhibiting apoptotic processes. Akt1 is also able to induce protein synthesis pathways, and is therefore a key signaling protein in the cellular pathways that lead to skeletal muscle hypertrophy, and general tissue growth. Mouse model with complete deletion of Akt1 manifests growth retardation and increased spontaneous apoptosis in tissues such as testes and thymus. Since it can block apoptosis, and thereby promote cell survival, Akt1 has been implicated as a major factor in many types of cancer. Akt (now also called Akt1) was originally identified as the oncogene in the transforming retrovirus, AKT8.

Akt2 is an important signaling molecule in the insulin signaling pathway. It is required to induce glucose transport. In a mouse which is null for Akt1 but normal for Akt2, glucose homeostasis is unperturbed, but the animals are smaller, consistent with a role for Akt1 in growth. In contrast, mice which do not have Akt2, but have normal Akt1, have mild growth deficiency and display a diabetic phenotype (insulin resistance), again consistent with the idea that Akt2 is more specific for the insulin receptor signaling pathway.

The Akt inhibitor can be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the disclosure.

The Akt inhibitor is an antibody or fragment thereof that inhibits Akt activity.

Akt inhibitors are well known in the art. Exemplary Akt inhibitors include for example, VQD-002, Perifosine. Miltefosine, MK-2206, AZD5363 and Ipatasertib.

### Mammalian Target of Rapamycin (mTOR) Inhibitors

mTOR inhibitors are a class of drugs that inhibit the mammalian target of rapamycin (mTOR), which is a serine/threonine-specific protein kinase that belongs to the family of phosphatidylinositol-3 kinase (PI3K) related kinases (PIKKs). mTOR regulates cellular metabolism, growth, and proliferation by forming and signaling through two protein complexes, mTORC1 and mTORC2. The most established mTOR inhibitors are so-called rapalogs (rapamycin and its analogs), which have shown tumor responses in clinical trials against various tumor types.

It appears that growth factors, amino acids, ATP, and oxygen levels regulate mTOR signaling. Several downstream pathways that regulate cell-cycle progression, translation, initiation, transcriptional stress responses, protein stability, and survival of cells are signaling through mTOR.

The serine/threonine kinase mTOR is a downstream effector of the PI3K/AKT pathway, and forms two distinct multiprotein complexes, mTORC1 and mTORC2. These two complexes have a separate network of protein partners, feedback loops, substrates, and regulators. mTORC1 consists of mTOR and two positive regulatory subunits, raptor and mammalian LST8 (mLST8), and two negative regulators, proline-rich AKT substrate 40 (PRAS40) and DEPTOR. mTORC2 consists of mTOR, mLST8, mSin1, protor, rictor, and DEPTOR.

The mTOR inhibitor can be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the disclosure.

The mTor inhibitor is an antibody or fragment thereof that inhibits mTor activity.

mTor inhibitors are well known in the art. First generation mTOR inhibitors included rapamycin, sirolimus, temsirolimus (CCI-779), everolimus (RAD001), and ridaforolimus (AP-23573).

Second generation mTOR inhibitors are known as ATP-competitive mTOR kinase inhibitors. mTORC1/mTORC2 dual inhibitors are designed to compete with ATP in the catalytic site of mTOR. They inhibit all of the kinase-dependent functions of mTORC1 and mTORC2 and therefore, block the feedback activation of PI3K/AKT signaling, unlike rapalogs that only target mTORC1. These types of inhibitors have been developed and several of them are being tested in clinical trials. Like rapalogs, they decrease protein translation, attenuate cell cycle progression, and inhibit angiogenesis in many cancer cell lines and also in human cancer.

The close interaction of mTOR with the PI3K pathway has also led to the development of mTOR/PI3K dual inhibitors. Compared with drugs that inhibit either mTORC1 or PI3K, these drugs have the benefit of inhibiting mTORC1, mTORC2, and all the catalytic isoforms of PI3K. Targeting both kinases at the same time reduces the upregulation of PI3K, which is typically produced with an inhibition on mTORC1.

### Methods for Enhancing Ex Vivo Expansion of CAR-T Cells

Typically, CAR-T cells are stimulated ex vivo for their expansion to have enough cells prior to in vivo infusion. This ex vivo process is critical to have sufficient CAR-T in relatively less differentiated stage. After the ex vivo clonal expansion CAR-T are infused back into patient circulation where they undergo the process of “trafficking” in which they are directed to their site of action. Trafficking to the site of action can be mediated through overexpression of chemokines or molecules that will direct the CAR-T to its target site. While treatment with CAR-T has shown promising results they have been shown to induce toxicity in patients through macrophage activation syndrome (MAS), cytokine release syndrome (CRS), tumor lysis syndrome (TLS) and autoimmune toxicity.

Macrophage activation syndrome denotes the condition where there is an increase in T cell expansion and elevated levels of macrophage activation in vivo.

Cytokine release syndrome or a “cytokine storm” is severe immune reaction in which the body releases too many cytokines into the blood too quickly. Cytokines play an important role in normal immune responses, but having a large amount of them released in the body all at once can be harmful. In vivo T cell clonal expansion and response to an antigen leads to the “cytokine storm”. The presence of high levels of cytokines results in an elevated immune response (e.g. B cells, NK cells, macrophages, PMNs), inflammation, and tissue damage. Particularly, IL-6 is commonly elevated during a cytokine storm and at high levels can lead to trans-signaling with soluble IL-6 receptor.

Tumor lysis syndrome involves massive tumor cell lysis that releases large amounts of the tumors’ intracellular contents into systemic circulation. This often leads to hyperkalemia, hyperuricemia, hypophosphatemia, and hypocalcemia.

Autoimmune toxicity or “on target, off tumor toxicity” occurs when the CAR-T attacks the correct antigen target, but the tissue is nonmalignant. The risk of autoimmune toxicity increases after treatment with checkpoint inhibitors. Possible solutions to reduce this toxicity is to include treatment with corticosteroids and by specific inhibitors of IL-6 signaling.

Current T cell expansion technologies only partially recapitulate the in vivo microenvironment found in the human lymph nodes. Typically, in the production of CART-T cells, the T-cells are activated under close cell-cell contact via antigen presenting cells (APCs) such as dendritic cells (DCs), which present peptide major histocompatibility complexs (MHCs) to T cells as well as a variety of other costimulatory signals. These close quarters allow for efficient autocrine/paracrine signaling among the expanding T cells, which secrete IL2 and other cytokines to assist their own growth. Additionally, the lymphoid tissues are comprised of extracellular matrix (ECM) components such as collagen, which provide signals to upregulate proliferation, cytokine production, and pro-survival pathways. A variety of solutions have been proposed to make the T cell expansion process more physiological. One strategy is to use modified feeder cell cultures to provide activation signals similar to those of DCs. While this has the theoretical capacity to mimic many components of the lymph node, it is hard to reproduce on a large scale due to the complexity and inherent variability of using cell lines in a fully Good Manufacturing Practices (GMP)-compliant manner. Others have proposed biomaterials-based solutions to circumvent this problem, including lipid-coated microrods, 3D-scaffolds via either Matrigel or 3d-printed lattices, ellipsoid beads, and mAb-conjugated polydimethylsiloxane (PDMS) beads that respectively recapitulate the cellular membrane, large interfacial contact area, 3D-structure, or soft surfaces T cells normally experience in vivo. While these have been shown to provide superior expansion compared to traditional microbeads, no method has been able to show preferential expansion of functional memory and CD4 T cell populations. Generally, T cells with a lower differentiation state such as memory cells have been shown to provide superior anti-tumor potency, presumably due to their higher potential to replicate, migrate, and engraft, leading to a long-term, durable response. Likewise, CD4 T cells are similarly important to anti-tumor potency due to their cytokine release properties and ability to resist exhaustion.

In some embodiments the CAR-T cells are stimulated ex vivo for their expansion prior to injecting in cancer patients. The process for efficient expansion of CAR-T cells requires activation of TCR complex and co-stimulation anti-CD3/anti-CD-28. Co-stimulation with anti-CD3 is important as it can bind to CD3 subunit to activate TCR complex without the need of antigenic peptide from the antigen presenting cells. Similarly, anti-CD28 can bind to CD28 and stimulates the T cells without the need for CD80 or CD86 from antigen presenting cells. Hence, co-stimulation with anti-CD3 and/or anti-CD28 is useful for CAR-T in vivo expansion.

For ex vivo expansion, it is required to provide a surface to which CAR-T cells can adhere and this can be achieve by using either plate or bead bound anti-CD3 to facilitate binding of the CD3 component of the T cell receptor on CAR-T cells, which is required to create a surface that mimics the immunological synapse. Similarly, CD28 is the essential costimulatory molecule required to drive naive T cell proliferation. Hence, anti-CD28 antibodies can either be added to the immobilized anti-CD3, i.e. plate or bead bound, or alternatively added in solution.

A critical consideration being ease of translation to industry and ability to interface with scalable systems such as bioreactors. It has been shown that using porous microcarriers functionalized with anti-CD3 and anti-CD28 mAbs in T cell expansion cultures. Microcarriers have historically been used throughout the bioprocess industry for adherent cultures such as stem cells and Chinese hamster ovary (CHO) cells, but not with suspension cells such as T cells. The macroporous structure in beads allows T cells to grow inside and along the surface, providing ample cell-cell contact for enhanced autocrine and paracrine signaling. Furthermore, the beads were composed of gelatin, which is a collagen derivative and therefore has adhesion domains that are also present within the lymph nodes. Finally, the 3D surface of the carriers provides a larger contact area for T cells, which may emulate the large contact surface area that occurs between T cells and DCs. It has been shown that traditional beads coated with anti-CD3/anti-CD28 not only provide superior CAR-T expansion, but consistently provide a higher frequency of memory and CD4 T cells (CCR7+CD62L+) across multiple donors. Thus, microbead coated with anti-CD3/anti-CD28 may be used along with specific inhibitors of PI3K-AKT-mTOR pathway and IL-6 signaling to enhance anti-tumor activity of CAR-T.

### Methods of Enhancing CAR-T Cell Therapy

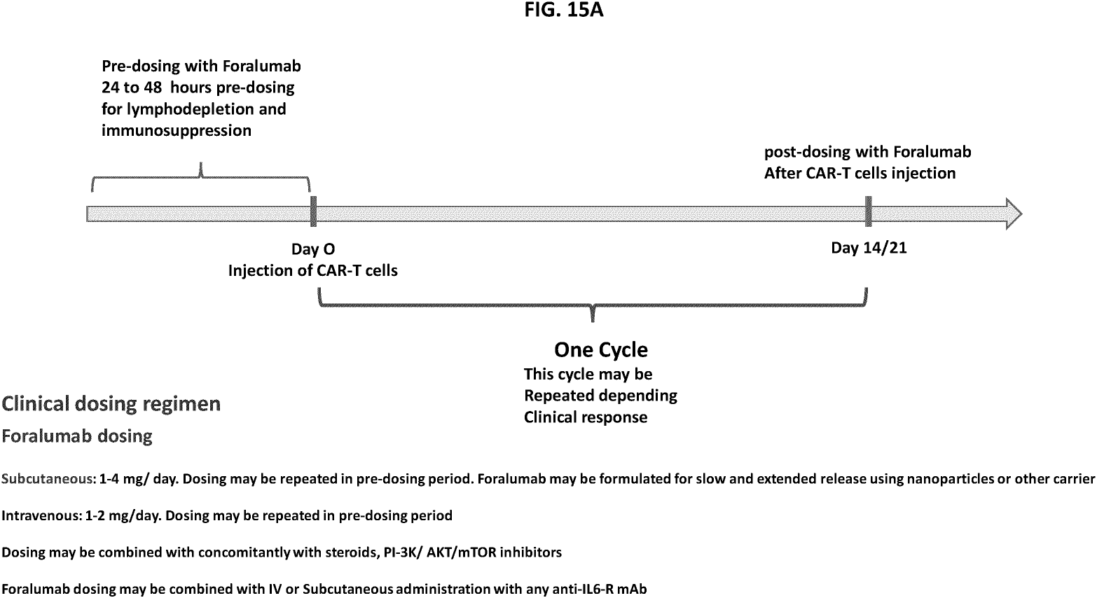

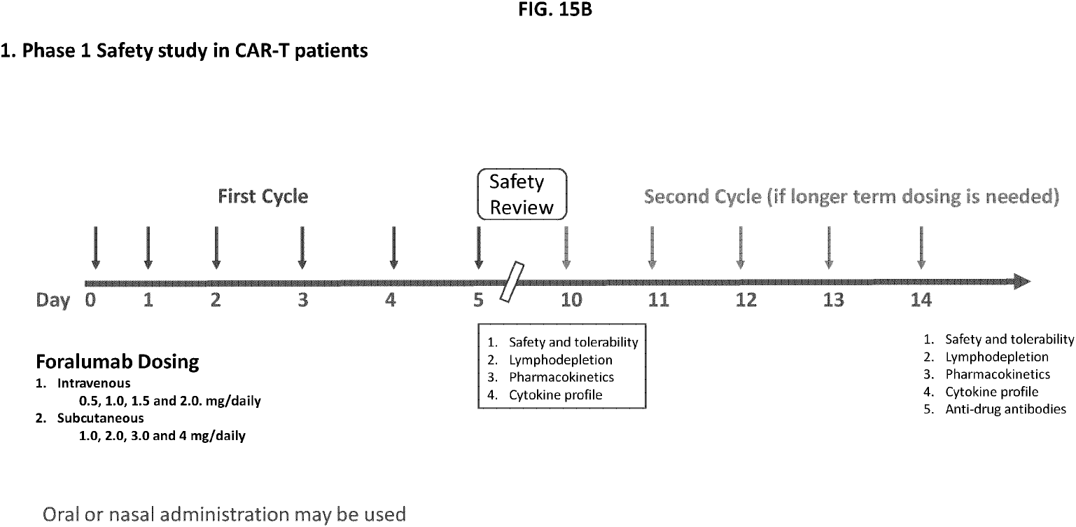

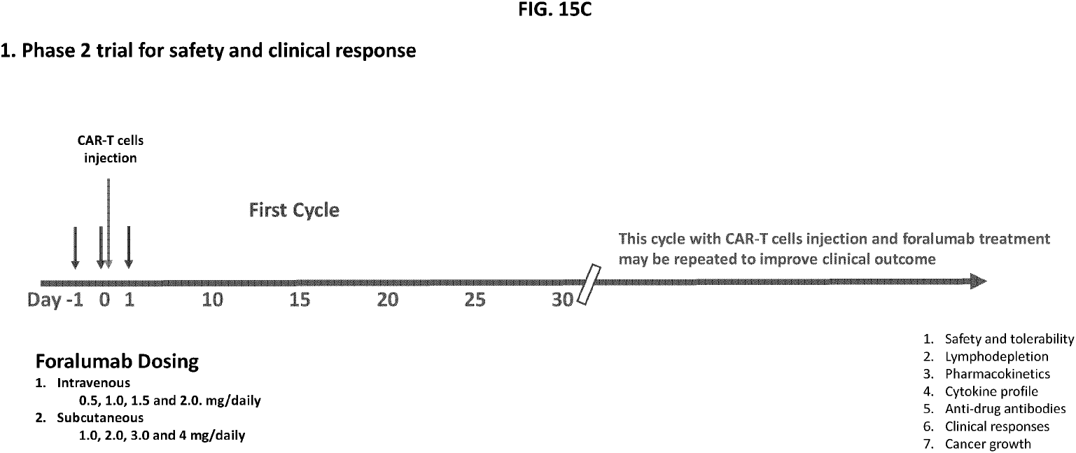

The disclosure provides dosing regimens for administering antibodies to enhance CAR-T therapy. Illustrative dosing regimens can be found in FIGS. 15A-C. The antibodies can be, for example, an antibody or fragment thereof targeting a T cell surface protein. In some embodiments, the antibody binds to a T cell surface protein can be a T cell activating protein. In some embodiments, the antibody binds to CD3 (i.e. anti-CD3 antibody). In some embodiments, the antibody can be any of the antibodies or fragments thereof described herein. In some embodiments, the antibody is foralumab.

The anti-CD3 antibody used in the methods of enhancing CAR-T cell therapy described herein can be administered in any way that achieves the desired outcome of enhancing CAR-T therapy. Enhancing CAR-T cell therapy can include, without limitation, achieving lymphodepletion, improved safety and tolerability, improved pharmacokinetics, and/or improved cytokine profile, reduced host versus graft disease, reduced graft versus host disease. In some embodiments, the anti-CD3 antibody is administered intravenously. In some embodiments, the anti-CD3 antibody is administered by subcutaneous injection. In some embodiments, the anti-CD3 antibody is administered orally. In some embodiments, the anti-CD3 antibody is administered nasally.

The anti-CD3 antibody used in the methods of enhancing CAR-T cell therapy described herein can be administered at any dose that achieves the desired outcome of enhancing CAR-T therapy (e.g. achieving lymphodepletion, improved safety and tolerability, improved pharmacokinetics, and/or improved cytokine profile). In some embodiments, the anti-CD3 antibody dose is about 0.5 mg/day, about 1.0 mg/day, about 1.5 mg/day, about 2.0 mg/day, about 2.5 mg/day, about 3.0 mg/day, about 3.5 mg/day, about 4 mg/day, about 5 mg/day, about 5.5 mg/day, about 6 mg/day, about 6.5 mg/day, about 7 mg/day, about 7.5 mg/day, about 8 mg/day, about 8.5 mg/day, about 9 mg/day, about 9.5 mg/day, or about 10 mg/day.

In one aspect, the methods described herein comprise enhancing CAR-T therapy comprising administering an antibody to a subject prior to administering a CAR-T cell composition, which herein can be referred to as “pre-dosing” or “first cycle.” In some embodiments, pre-dosing causes lymphodepletion and/or immunosuppression in the subject. Lymphodepletion and immunosuppression can enhance CAR-T therapy by promoting the survival of engineered CAR-T cells in vivo and reduction of severe toxic effects observed in CAR-T therapy. In some embodiments, pre-dosing reduces host versus graft disease. In some embodiments, pre-dosing improves safety and tolerability of treatment with CAR-T cells. In some embodiments, pre-dosing improves pharmacokinetics of CAR-T therapy. In some embodiments, pre-dosing improves the cytokine profile associated with CAR-T therapy. In some embodiments, the pre-dosing is performed about 8 hours, 16 hours, 24 hours, 32 hours, 40 hours, 48 hours, 56 hours, 64 hours, 72 hours, 80 hours, 88 hours, 96 hours, 104 hours, 112 hours, or 120 hours prior to administration of a CAR-T cell composition. In some embodiments, pre-dosing is performed one or more times prior to administration of a CAR-T cellular composition. In some embodiments, pre-dosing is repeated until lymphodepletion and/or immunosuppression is confirmed.

In one aspect, the methods described herein comprise administering a CAR-T cell composition to a subject following pre-dosing. The CAR-T cell composition can be any CAR-T cell composition. The CAR-T cell composition can be derived from, for example, autologous cells or allogeneic T cells. The CAR-T cell composition can be directed toward any target. The target can be, for example, a tumor associated antigen or a pathogenic antigen. The CAR-T cell composition can be administered by intravenous injection. Response to administration of the CAR-T cell composition can be monitored continuously or at specific time points. In some embodiments, an anti-CD3 antibody is co-administered with the CAR-T cell composition. In some embodiments, response to administration of a CAR-T composition is monitored at day 7, day 10, day 12, day 13, day 14, day 21, or day 28 following administration of the CAR-T cell composition. It is appreciated in the art that the specific measurement of response to an administered CAR-T cell composition will be determined by the type of CAR-T cell composition, the CAR-T cell therapy indication, needs of the subject receiving the CAR-T cell composition, and the qualified medical professionals responsible for treating the subject. The measured response can include, for example, an assessment of toxic or adverse events in the subject. The measured response can include, for example, an assessment of disease progression in the subject. The measured response can include, for example, an assessment of the presence of a pathogen in the subject. In some embodiments, administration the CAR-T cell composition to a subject will be repeated one or more times. In some embodiments, a CAR-T cell composition will be administered to a subject the repeatedly to reach a pre-determined measured response.

In one aspect, the methods described herein comprise administering an anti-CD3 antibody following administration of the CAR-T cell composition. Enhancing CAR-T therapy by administering an antibody to a subject following administering a CAR-T cell composition, can be called “post-dosing” or “second cycle.” In some embodiments, post-dosing causes lymphodepletion and/or immunosuppression in the subject. Lymphodepletion and immunosuppression can enhance CAR-T therapy by promoting the survival of engineered CAR-T cells in vivo and reduction of severe toxic effects observed in CAR-T therapy. In some embodiments, post-dosing improves safety and tolerability of treatment with CAR-T cells. In some embodiments, post-dosing improves pharmacokinetics of CAR-T therapy. In some embodiments, post-dosing improves the cytokine profile associated with CAR-T therapy. In some embodiments, post-dosing is part of a regimen that includes co-administration of anti-drug antibody. For example, an antibody or small molecule drug that depletes the CAR-T cell composition. In some embodiments, the post-dosing is performed at day 7, day 14, day 21, or day 28 following administration of the CAR-T cell composition. In some embodiments, post-dosing is performed one or more times following administration of a CAR-T cellular composition. In some embodiments, post-dosing is repeated until lymphodepletion and/or immunosuppression is confirmed.

In some embodiments, the antibody administered prior to or following a CAR-T cell composition is an anti-CD3 antibody. In some embodiments, the antibody administered prior to or following a CAR-T cell composition is an antibody described herein. In some embodiments, the antibody administered prior to or following a CAR-T cell composition is foralumab. In some embodiments, the antibody administered prior to or following a CAR-T cell composition is delivered intravenously. In some embodiments, the antibody administered in pre-dosing or post-dosing is delivered by subcutaneous injection. In some embodiments, the antibody administered prior to or following a CAR-T cell composition is delivered as a pharmaceutical composition comprising foralumab and a pharmaceutically acceptable carrier. The pharmaceutical composition can be a pharmaceutical compositions described herein. In some embodiments, the pharmaceutical composition comprises a unit dose of antibody. In some embodiments, the unit dose is 1, 2, 3, or 4 mg of antibody. In some embodiments, the pharmaceutical composition is formulated for slow and extended release using a carrier. In some embodiments, the carrier is a nanoparticle.

The antibody or pharmaceutical composition administered in pre-dosing or post-dosing can be combined with one or more additional agents. In some embodiments, the antibody or pharmaceutical composition is administered in combination with a steroid. In some embodiments, the antibody or pharmaceutical composition is administered in combination with a PI3K inhibitor. In some embodiments, the antibody or pharmaceutical composition is administered in combination with an ATK inhibitor. In some embodiments, the antibody or pharmaceutical composition is administered in combination with an mTOR inhibitor. In some embodiments, the antibody or pharmaceutical composition is administered with an anti-IL6R antibody.

### Methods of Enhancing Cell Therapies

In one aspect, the disclosure provides methods of enhancing cell therapies. Cell therapies can be any therapy comprising transplanting (i.e. administering) of cells for the treatment of a subject in need thereof. In some embodiments, the cell therapy comprises allogeneic cell therapy. In some embodiments, the cell therapy comprises autologous cell therapy. In some embodiments, the cell therapy comprises xenogeneic cell therapy.

The cell therapies contemplated herein may comprise any cell type appropriate for the treatment of a subject in need thereof. In some embodiments, the cell therapy comprises stem cells. In some embodiments, the stem cell is a human embryonic stem cell. In some embodiments, the stem cell is a tissue-specific stem cell. In some embodiments, the stem cell is a neural stem cell. In some embodiments, the stem cell is a mesenchymal stem cell. In some embodiments, the stem cell is a hematopoietic stem cells. In some embodiments, the stem cell is an induced pluripotent stem cell. In some embodiments, the stem cell is an epidermal stem cell. In some embodiments, the stem cell is an epithelial stem cell. In some embodiments, the stem cell is a neural stem cell. In some embodiments, the cell therapy comprises differentiated or mature cells. In some embodiments, the cell therapy comprises immune cells. In some embodiments, the immune cell is a lymphocyte. In some embodiments, the immune cell is a T lymphocyte. In some embodiments, the immune cell is a B cell. In some embodiments, the immune cell is a regulatory T cell. In some embodiments, the immune cell is a CD4+ T cell. In some embodiments, the immune cell is a CD8+ T cell. In some embodiments, the immune cell is a hyper T cell. In some embodiments, the immune cell is a cytotoxic T cell. In some embodiments, the immune cell is a natural killer cell. In some embodiments, the immune cell is a NKT cell.

In some embodiments, the cell therapy comprises an engineered cell. An engineered cell can be a autologous, allogeneic, or artificial cell that comprises an exogenous polynucleotide. For example, an engineered cell may contain exogenous, or foreign, polynucleotides delivered to the cell by transfection or transduction. Transfection or transduction can be accomplished using any method known in the art to deliver exogenous polynucleotides to a cell. For example, plasmid polynucleotides can be delivered using, for example, electroporation or lipid-based transfection reagents (e.g. lipofectamine). Engineered cells may be infected, or transduced, with a viral vector that delivers the exogenous polynucleotide. Transduction can be accomplished, for example, by delivering an exogenous polynucleotide to a cell using a viral vector, such as a lentivirus, adenovirus, or adeno-associated virus. Engineered cells may be stably or transiently transfected. Methods of manipulating polynucleotides and vectors for use in delivering exogenous polynucleotides to cells are generally known in the art (e.g. Sambrook. Molecular cloning : a laboratory manual. Cold Spring Harbor Laboratory Press Cold Spring Harbor, N.Y 2012).

The exogenous polynucleotide can include transcriptional regulatory elements (e.g. promoters or enhancers). The exogenous polynucleotide can encode a product expressed by the engineered cell. The transcriptional regulatory element may be operably linked, i.e. controls expression, to a product encoded by the exogenous polynucleotide. The exogenous polynucleotide can include a non-coding RNA product, such as an interfering RNA or a guide RNA for a targeted nuclease system. The exogenous polynucleotide can include a product that encodes a polypeptide.

Engineered cells can express a polypeptide encoded by an exogenous polynucleotide. Expression of the polypeptide can be inducible, repressible, or constitutive. The polypeptide can be, without limitation, an artificial receptor, a chimeric antigen receptor, an engineered T cell receptor, a fusion protein, a factor that promotes survival of the engineered cell, a suicide gene, a cytokine, or any polypeptide that enhances safety or efficacy of a cell therapy.

Engineered cells can have one or more modifications to an endogenous gene. Modifications to an endogenous gene can be accomplished using any gene editing method known in the art. For example, common gene editing methods include targeted nuclease systems such as CRISPR/Cas, TALEN, and zinc-finger nucleases. Modifications can be, for example, mutations (e.g. deletions) targeted to a specific gene or transcriptional regulatory element operably linked to a specific gene. The specific gene can be, for example, an immune-related gene such as a gene encoding a polypeptide in the major histocompatibility complex (e.g. beta-2-microglobulin or a human leukocyte antigen). The gene can have a functional role in the safety and efficacy of a cell therapy. For example, the functional role can be modulation of host versus graft or graft versus host disease.

The disclosure provides dosing regimens for administering antibodies to enhance cell therapy. Illustrative dosing regimens can be found in FIGS. 15A-C. The antibodies can be, for example, an antibody or fragment thereof targeting a T cell surface protein. In some embodiments, the antibody binds to a T cell surface protein can be a T cell activating protein. In some embodiments, the antibody binds to CD3 (i.e. anti-CD3 antibody). In some embodiments, the antibody can be any of the antibodies or fragments thereof described herein. In some embodiments, the antibody is foralumab.

The anti-CD3 antibody used in the methods of enhancing cell therapy described herein can be administered in any way that achieves the desired outcome of enhancing cell therapy. Enhancing cell therapy can include, without limitation, achieving lymphodepletion, improved safety and tolerability, improved pharmacokinetics, and/or improved cytokine profile, reduced host versus graft disease, reduced graft versus host disease. In some embodiments, the anti-CD3 antibody is administered intravenously. In some embodiments, the anti-CD3 antibody is administered by subcutaneous injection. In some embodiments, the anti-CD3 antibody is administered orally. In some embodiments, the anti-CD3 antibody is administered nasally.

The anti-CD3 antibody used in the methods of enhancing cell therapy described herein can be administered at any dose that achieves the desired outcome of enhancing cell therapy (e.g. reduced graft versus host disease, reduced host versus graft disease, achieving lymphodepletion, improved safety and tolerability, improved pharmacokinetics, and/or improved cytokine profile). In some embodiments, the anti-CD3 antibody dose is about 0.5 mg/day, about 1.0 mg/day, about 1.5 mg/day, about 2.0 mg/day, about 2.5 mg/day, about 3.0 mg/day, about 3.5 mg/day, about 4 mg/day, about 5 mg/day, about 5.5 mg/day, about 6 mg/day, about 6.5 mg/day, about 7 mg/day, about 7.5 mg/day, about 8 mg/day, about 8.5 mg/day, about 9 mg/day, about 9.5 mg/day, or about 10 mg/day.

In one aspect, the methods described herein comprise enhancing cell therapy comprising administering an antibody to a subject prior to administering a cell therapy composition, which herein can be referred to as “pre-dosing” or “first cycle.” In some embodiments, pre-dosing causes lymphodepletion and/or immunosuppression in the subject. Lymphodepletion and immunosuppression can enhance cell therapy by promoting the survival of transplanted cells in vivo and reduction of severe toxic effects observed in cell therapy. In some embodiments, pre-dosing improves safety and tolerability of treatment with transplanted cells. In some embodiments, pre-dosing improves pharmacokinetics of cell therapy. In some embodiments, pre-dosing improves the cytokine profile associated with cell therapy. In some embodiments, the pre-dosing is performed about 8 hours, 16 hours, 24 hours, 32 hours, 40 hours, 48 hours, 56 hours, 64 hours, 72 hours, 80 hours, 88 hours, 96 hours, 104 hours, 112 hours, or 120 hours prior to administration of a cell therapy composition. In some embodiments, pre-dosing is performed one or more times prior to administration of a cell therapy composition. In some embodiments, pre-dosing is repeated until lymphodepletion and/or immunosuppression is confirmed.

In one aspect, the methods described herein comprise administering a cell therapy composition to a subject following pre-dosing. The cell therapy composition can be any cell therapy composition. In some embodiments, an anti-CD3 antibody is co-administered with the cell therapy composition. In some embodiments, response to administration of a cell therapy composition is monitored at day 7, day 10, day 12, day 13, day 14, day 21, or day 28 following administration. It is appreciated in the art that the specific measurement of response to an administered cell therapy composition will be determined by the type of cell therapy composition, the cell therapy indication, needs of the subject receiving the cell therapy composition, and the qualified medical professionals responsible for treating the subject. The measured response can include, for example, an assessment of toxic or adverse events in the subject. The measured response can include, for example, an assessment of disease progression in the subject. The measured response can include, for example, an assessment of the presence of a pathogen in the subject. In some embodiments, administration the cell therapy composition to a subject will be repeated one or more times. In some embodiments, a cell therapy composition will be administered to a subject repeatedly to reach a pre-determined measured response.

In one aspect, the methods described herein comprise administering an anti-CD3 antibody following administration of the cellular thearpy composition. Enhancing cell therapy by administering an antibody to a subject following administering a cell therapy composition, can be called “post-dosing” or “second cycle.” In some embodiments, post-dosing causes lymphodepletion and/or immunosuppression in the subject. Lymphodepletion and immunosuppression can enhance cell therapy by promoting the survival of administered or transplanted cells in vivo and reduction of severe toxic effects observed in cell therapy. In some embodiments, post-dosing improves safety and tolerability of treatment with cell therapy. In some embodiments, post-dosing improves pharmacokinetics of cell therapy. In some embodiments, post-dosing improves the cytokine profile associated with cell therapy. In some embodiments, post-dosing is part of a regimen that includes co-administration of anti-drug antibody. For example, an antibody or small molecule drug that depletes, maintains, or promotes persistence of the cell therapy composition. In some embodiments, the post-dosing is performed at day 7, day 14, day 21, or day 28 following administration of the cell therapy composition. In some embodiments, post-dosing is performed one or more times following administration of a cell therapy composition. In some embodiments, post-dosing is repeated until lymphodepletion and/or immunosuppression is confirmed.

In some embodiments, the antibody administered prior to or following a cell therapy composition is an anti-CD3 antibody. In some embodiments, the antibody administered prior to or following a CAR-T cell composition is an antibody described herein. In some embodiments, the antibody administered prior to or following a CAR-T cell composition is foralumab. In some embodiments, the antibody administered prior to or following a cell therapy composition is delivered intravenously. In some embodiments, the antibody administered in pre-dosing or post-dosing is delivered by subcutaneous injection. In some embodiments, the antibody administered prior to or following a cell therapy composition is delivered as a pharmaceutical composition comprising foralumab and a pharmaceutically acceptable carrier. The pharmaceutical composition can be a pharmaceutical compositions described herein. In some embodiments, the pharmaceutical composition comprises a unit dose of antibody. In some embodiments, the unit dose is 1, 2, 3, or 4 mg of antibody. In some embodiments, the pharmaceutical composition is formulated for slow and extended release using a carrier. In some embodiments, the carrier is a nanoparticle.

The antibody or pharmaceutical composition administered in pre-dosing or post-dosing can be combined with one or more additional agents. In some embodiments, the antibody or pharmaceutical composition is administered in combination with a steroid. In some embodiments, the antibody or pharmaceutical composition is administered in combination with a PI3K inhibitor. In some embodiments, the antibody or pharmaceutical composition is administered in combination with an ATK inhibitor. In some embodiments, the antibody or pharmaceutical composition is administered in combination with an mTOR inhibitor. In some embodiments, the antibody or pharmaceutical composition is administered with an anti-IL6R antibody.,

### Pharmaceutical Compositions

The CD-3 antibodies, IL-6Rc antibodies, CD28 antibodies, PI3K inhibitors, Akt inhibitors and the mTor inhibitors. (also referred to herein as “active compounds”), and derivatives, fragments, analogs and homologs thereof, are incorporated into pharmaceutical compositions suitable for administration (“also referred to herein therapeutic compositions”).

The active compounds may be formulated in the same therapeutic composition, when it is desired that the active compounds be administered together and by the same route of administration. Alternatively, active compounds may be formulated in the different therapeutic compositions. This is useful when it is desired that the active compounds be administered separately and/or by different routes of administration

Principles and considerations involved in preparing such pharmaceutical compositions, as well as guidance in the choice of components are provided, for example, in Remington’s Pharmaceutical Sciences: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)). The active compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The active compounds will be present in such compositions in amounts sufficient to provide the desired dosage amount in the range described herein.

The formulations to be used for ex vivo use and in vivo administration must be sterile. This is readily accomplished by filtration through sterile filtration membranes.

Pharmaceutical compositions of the disclosure may be administered orally, nasally, transdermally, pulmonary, inhalationally, buccally, sublingually, intraperitoneally, subcutaneously, intramuscularly, intravenously, rectally, intrapleurally, intrathecally and parenterally. One skilled in the art will recognize the advantages of certain routes of administration.

Active compounds may be formulated to included carriers or diluents include, such as but are not limited to, water, saline, ringer’s solutions, dextrose solution, and 5% human serum albumin. Liposomes and non-aqueous vehicles such as fixed oils may also be used.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor® EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze drying that yields a powder of the active ingredient plus any additional desired ingredient from a previously sterile filtered solution thereof.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The composition, formulation, or pharmaceutically acceptable composition contain excipients such as stabilizers, preservative, phospholipids and/or other ingredients to improve stability and shelf life and in the case of dactinomycin nanoparticles a uniform particle size. Exemplary excipients include, but are not limited to Trehalose (1-20%), a surfactant, sodium chloride (50-150 mM), EDTA or EGTA (0.1 to 1 mM), a buffer such as sodium citrate buffer (10-50 mM).

For administration by inhalation, the active compounds are delivered in the form of an aerosol spray from pressured container or dispenser, which contains a suitable propellant, e.g., a gas such as carbon dioxide, or a nebulizer.

Administration by inhalation may be in the form of an inhaler or a nebulizer. The nebulizer and/or inhaler is handheld. Optionally, the nebulizer and/or inhaler can be of different sizes to fit children and/or adults.

In some embodiments, a vial containing a stabilized and formulated solution the active compounds described herein is inserted into an inhaler and/or nebulizer. In some embodiments, a vial containing a stabilized and formulated solution of active compounds described herein is inserted into the bottom of the inhaler and/or nebulizer. In some embodiments, the pharmaceutical composition is dispensed as fine aerosols through the mouth.

The compounds can also be prepared in the form of suppositories (e.g., with conventional suppository bases such as cocoa butter and other glycerides) or retention enemas for rectal delivery.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethylmethacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(^{_})-3-hydroxybutyric acid. While polymers such as ethylene-vinyl acetate and lactic acid-glycolic acid enable release of molecules for over 100 days, certain hydrogels release proteins for shorter time periods.

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer’s specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the term “antibody” refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, Fab, Fab’ and F(ab′)2 fragments, and an Fab expression library. By “specifically bind” or “immunoreacts with” is meant that the antibody reacts with one or more antigenic determinants of the desired antigen and does not react (i.e., bind) with other polypeptides or binds at much lower affinity (Kd > 10-6) with other polypeptides.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody’s isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ea., 2nd ed. Raven Press, N.Y. (1989)). The variable regions of each light/heavy chain pair form the antibody binding site.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG1, IgG2, and others. Furthermore, in humans, the light chain may be a kappa chain or a lambda chain.

As used herein, the term “epitope” includes any protein determinant capable of specific binding to an immunoglobulin, a scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≤ 1 µM; preferably ≤ 100 nM and most preferably ≤ 10 nM.

As used herein, the terms “immunological binding” and “immunological binding properties” and “specific binding” refer to the non-covalent interactions of the type which occur between an immunoglobulin molecule and an antigen for which the immunoglobulin is specific. The strength, or affinity of immunological binding interactions can be expressed in terms of the dissociation constant (Kd) of the interaction, wherein a smaller Kd represents a greater affinity. Immunological binding properties of selected polypeptides are quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kon) and the “off rate constant” (Koff) can be determined by calculation of the concentrations and the actual rates of association and dissociation. (See Nature 361: 186-87 (1993)). The ratio of Koff /Kon enables the cancellation of all parameters not related to affinity, and is equal to the dissociation constant Kd. (See, generally, Davies et al. (1990) Annual Rev Biochem 59:439-473). An antibody of the present disclosure is said to specifically bind to a CD3 epitope when the equilibrium binding constant (Kd) is about 1 µM, preferably about 100 nM, more preferably about 10 nM, and most preferably about 100 pM to about 1 pM, as measured by assays such as radioligand binding assays or similar assays known to those skilled in the art.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide- containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur- containing side chains is cysteine and methionine. Preferred conservative amino acids substitution groups are: valine-leucine-isoleucine, phenylalanine-tyrosine, lysine-arginine, alanine valine, glutamic- aspartic, and asparagine-glutamine.

As discussed herein, minor variations in the amino acid sequences of antibodies or immunoglobulin molecules are contemplated as being encompassed by the present disclosure, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family.

The term “agent” is used herein to denote a chemical compound, a mixture of chemical compounds, a biological macromolecule, or an extract made from biological materials.

The term patient includes human and veterinary subjects.

The disclosure also includes Fv, Fab, Fab′ and F(ab′)2 anti-CD3 antibody fragments, single chain anti-CD3 antibodies, bispecific anti-CD3 antibodies, heteroconjugate anti-CD3 antibodies, trispecific antibodies, immunoconjugates and fragments thereof.

Bispecific antibodies are antibodies that have binding specificities for at least two different antigens. In the present case, one of the binding specificities is for CD3. The second binding target is any other antigen, and advantageously is a cell-surface protein or receptor or receptor subunit.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The disclosure having now been described by way of written description, those of skill in the art will recognize that the disclosure can be practiced in a variety of embodiments and that the foregoing description and examples below are for purposes of illustration and not limitation of the claims that follow.

### EXAMPLES

### Example 1: Bioavailability Study of Foralumab Administered by Subcutaneous Delivery in a Mouse Model

The objective of this study was to compare the pharmacokinetic (PK) profiles of intravenous and subcutaneous administration of foralumab in a mouse model.

The results of this study will provide feasibility for administering foralumab via subcutaneous injection, which represents a possible human therapeutic route. The intravenous route of administration will be used as a comparator because it was validated in previous pre-clinical repeated dose toxicity studies and early clinical studies for administering foralumab.

### Study Design

The mouse model used in this study was the human CD3 epsilon transgenic mouse model, which harbors a humanized CD3 epsilon chain of the CD3 co-receptor within a functional mouse immune system. This model is used to determine the in vivo efficacy of human-specific immunotherapies that target the human CD3 epsilon chain. A total of 132 mice (66 males and 66 females) were used. Groups were dosed either intravenously (IV) or by subcutaneous injection (SC). Study groups 1, 2 and 3 were composed of 18 males and 18 females, divided into groups of 3 for each time point. Intravenous (IV) group 4 had 3 males and 3 females and the subcutaneous placebo group 5 had 9 males and 9 females. For each time point, 3 males and 3 females were used as described below (Table 1.1).

TABLE 1.1

Study Groups

| Group Number | Control / Test Article | Route of Injection | Gender | Time Points (hour) | | | | | |
|---|---|---|---|---|---|---|---|---|---|
| | | | | 0.5 | 2 | 6 | 24 | 48 | 120 |
| 1 | NI-0401 | IV | Males | 3 | 3 | 3 | 3 | 3 | 3 |
| | | | Females | 3 | 3 | 3 | 3 | 3 | 3 |
| 2 | NI-0401 | SC 1x | Males | 3 | 3 | 3 | 3 | 3 | 3 |
| | | | Females | 3 | 3 | 3 | 3 | 3 | 3 |
| 3 | NI-0401 | SC 2x | Males | 3 | 3 | 3 | 3 | 3 | 3 |
| | | | Females | 3 | 3 | 3 | 3 | 3 | 3 |
| 4 | Placebo | IV | Males | - | - | - | 3 | - | - |
| | | | Females | - | - | - | 3 | - | - |
| 5 | Placebo | SC | Males | - | - | - | 3 | 3 | 3 |
| | | | Females | - | - | - | 3 | 3 | 3 |

### Dose Formulation

Foralumab (NI-0401) was formulated in 25 mM sodium acetate buffer, 125 mM sodium chloride with 0.02% polysorbate 80, pH 5.5. The vehicle control (Placebo) used was 25 mM sodium acetate buffer, 125 mM sodium chloride with 0.02% polysorbate 80, pH 5.5.

### Dose Level and Volume

A single dose of 0.3 mg/kg was selected which was shown previously to cause up to 70% reduction of T cells in peripheral blood and 80% modulation of human CD3 epsilon molecules at the T cell membrane in LCD3 transgenic mice. Dose volume was 2.5 mL/kg manually injected as a bolus.

### Test Article Administration

A single dose of either the foralumab formulation or placebo was administered subcutaneously in the lateral side of the abdominal wall or else intravenously via the retro-orbital sinus.

### Blood Sampling

Blood samples were collected from the mice by intracardiac puncture under terminal anaesthesia at the indicated time points following administration. Samples were collected in Plasma Separator Tubes (BD), and the plasma separated by centrifugation. Aliquots of 40-50 µL plasma were frozen and stored at -50o C.

### Results and Conclusion

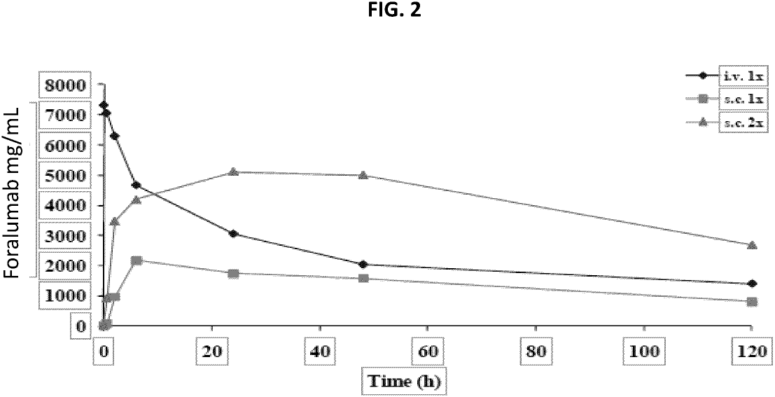

Subcutaneous administration of foralumab achieves efficient delivery into the blood and is pharmacologically active (FIG. 2, Table 1.2). Blood levels of subcutaneously delivered foralumab peaked between 6 and 24 hours following delivery (Table 1.3). Bioavailability of foralumab at 0.3 mg/kg delivered subcutaneously is approximately 55% (Table 1.2). Increasing the dose of foralumab to 0.6 mg/kg in a subcutaneous delivery increases the bioavailability to 92% (Table 1.2). Infusion related reactions are likely to be reduced because of a 50% reduction in Cmax achieved through subcutaneous compared to intravenous administration. Together these results suggest that delivery of foralumab by subcutaneous administration is a valid method for dosing subjects foralumab and related antibodies.

TABLE 12

Pharmacokinetic Parameters
Non Compartmental Analysis

| Dose (mg/kg) | Dose Route | Tmax (h) | Cmax (ng/mL) | AUC (0-t) (ng.h/mL) | AUC (0-inf.) (ng.h/mL) | λz (/hr) | Half-life (h) | No. Points | Bioavail (%) | MRT (h) |
|---|---|---|---|---|---|---|---|---|---|---|
| 0.3 | IV 1x | 0.0 | 7306 | 289439 | 476318 | 0.00749 | 93 | 3 | 100 | 127 |
| 0.3 | SC 1x | 6 | 2167 | 167001 | 261271 | 0.0085 | 81 | 4 | 55 | 118 |
| 0.6 | SC 2x | 24 | 5117 | 500505 | 873420 | 0.0072 | 96 | 3 | 92 | 142 |

TABLE 13

Plasma Concentrations from Different Routes of Administration

| PK Time (h) | 0.3 mg/kg IV 1x | 0.3 mg/kg SC 1x | 0.6 mg/kg SC 2X |
|---|---|---|---|
| 0 | 7306 | 0 | 0 |
| 0.5 | 7040 | 70 | 913 |
| 2 | 6283 | 950 | 3467 |
| 6 | 4660 | 2167 | 4183 |
| 24 | 3050 | 1733 | 5117 |
| 48 | 2033 | 1567 | 5000 |
| 120 | 1400 | 802 | 2683 |

### Example 2: Pharmakokinetic, Pharmacodynamics, and Safety Studies of Foralumab (NI-0401) by Intravenous Administration

The primary objective of these studies were to assess the safety and tolerability of foralumab in human subjects. The studies include an assessment of the pharmacokinetic profile, immunogenicity, and pharmacodynamic effects of foralumab delivered by intravenous administration for five days.

### Test Product, Dose, and Mode of Administration

The foralumab human monoclonal antibody was supplied in 3 mL vials, each containing 2 mL of the foralumab formulation at a concentration of 2.0 mg/mL. Each vial contained 4.0 mg of foralumab. The dose of foralumab was administered by intravenous infusion over 2 hours. The dosing schedules of foralumab were provided for eight different cohorts according to Table 2.1.

TABLE 21

Dosing Schedule

| Cohort | Day 1 (µg/m2 of Body Surface Area) | Day 2 (µg/m2 of Body Surface Area) | Day 3 (µg/m2 of Body Surface Area) | Day 4 (µg/m2 of Body Surface Area) | Day 5 (µg/m2 of Body Surface Area) |
|---|---|---|---|---|---|
| Cohort 1 | 500 | 500 | 500 | 500 | 500 |
| Cohort 2 | 500 | 500 | 650 | 650 | 650 |
| Cohort 3 | 650 | 650 | 650 | 650 | 650 |
| Cohort 4 | 650 | 650 | 1000 | 1000 | 1000 |
| Cohort 5 | 1000 | 1000 | 1000 | 1000 | 1000 |
| Cohort 6 | 1250 | 1250 | 1250 | 1250 | 1250 |
| Cohort 7 | 1500 | 1500 | 1500 | 1500 | 1500 |
| Cohort 8 | 1750 | 1750 | 1750 | 1750 | 1750 |

Pharmacokinetic profiles were determined by measuring foralumab blood plasma levels at the indicated times following administration. Foralumab plasma levels were measured using a ligand-binding assay. A specific anti-foralumab antibody was used as capture reagent and a fluorescently labeled anti-human IgG1. The sensitivity of the assay was 20 ng/mL (lower limit of detection).

### Pharmacokinetic Results

Foralumab plasma levels were measured using a ligand-binding assay. A specific anti-foralumab antibody was used as capture reagent and a fluorescently labeled anti-human IgG1. The sensitivity of the assay was 20 ng/mL (lower limit of detection).

For the study design in Table 2.1, the Cmax and AUC0-t parameters on Study Day 5 yielded mean values of 584.4 ng/mL (range 28.43 to 1860.1 ng/mL) and 28570 ng.h/mL (range 1453 to 106494 ng.h/mL), respectively across the dose range of 0.73 to 3.73 mg.

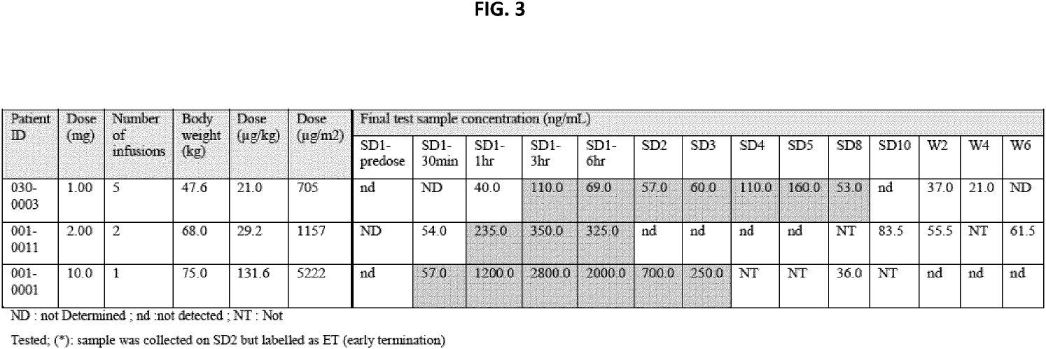

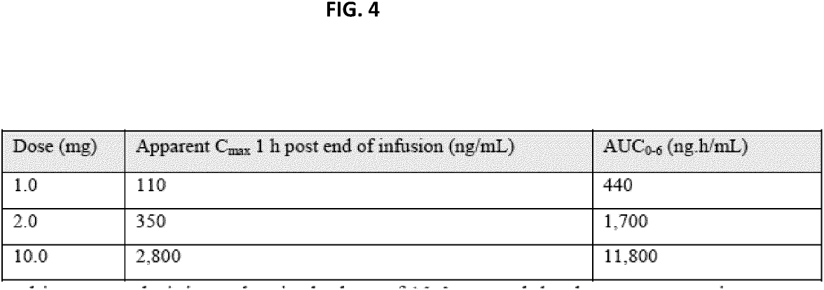

A representative sampling of pharmacokinetic profiles for three individuals from a separate study is shown in FIG. 3. These subjects were treated either with five doses of 1.0 mg (approximately +/- 500 µg/m2), two doses of 2.0 mg (approximately +/- 1000 µg/m2), or a single dose of 10.0 mg (approximately +/- 5000 µg/m2). For these 3 subjects, the concentration profiles were adequate to estimate apparent Cmax (1 hour post infusion) and AUC0-6, both showing a clear increase with dose (FIG. 4). The apparent Cmax values for the 1.0 mg, 2.0 mg, and 10.0 mg doses were 110 ng/mL, 350 ng/mL, and 2800 ng/mL, respectively. The AUC values for the 1.0 mg, 2.0 mg, and 10.0 mg doses were 440 ng.h/mL, 1700 ng.h/mL, and 11800 ng.h/mL, respectively.

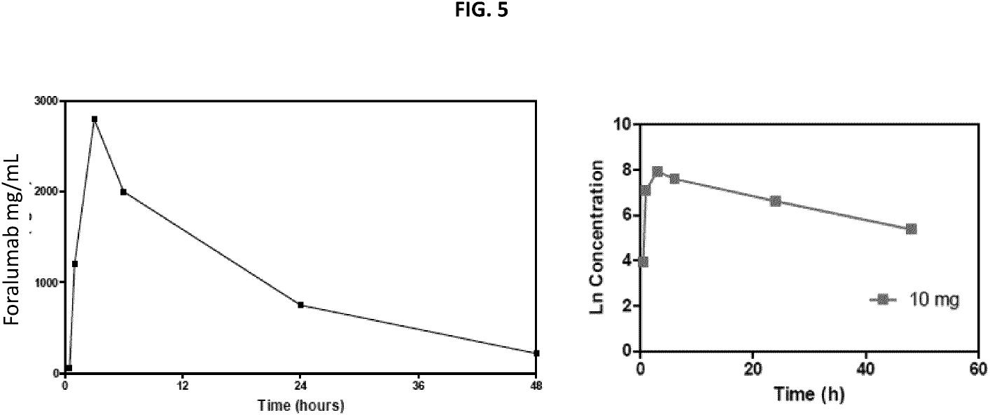

Graphs summarizing foralumab PK data obtained after a single 2h-infusion of 10.0 mg (subject 001-0001 in FIG. 4) are presented in FIG. 5. Following intravenous infusion of 10.0 mg study drug over 2 hours, the plasma concentrations of foralumab increased rapidly during the infusion period as expected. Post infusion, the concentration values declined in an essentially mono exponential manner. The early rapid decline in plasma concentration is due to binding of the drug to its target on circulating T-cells as well as drug distribution. The estimated plasma terminal half-life after a single dose of 10.0 mg foralumab was about 13h although with a more sensitive assay the terminal half-life may be considerably longer than this (theoretically it would take approximately 78 h to be eliminated from the systemic circulation, this being 6 times the terminal half-life). The measured half-life of foralumab is shorter than expected for an IgG1 molecule (usually approximately 3 weeks) because of the rapid uptake of the drug by the target cells.

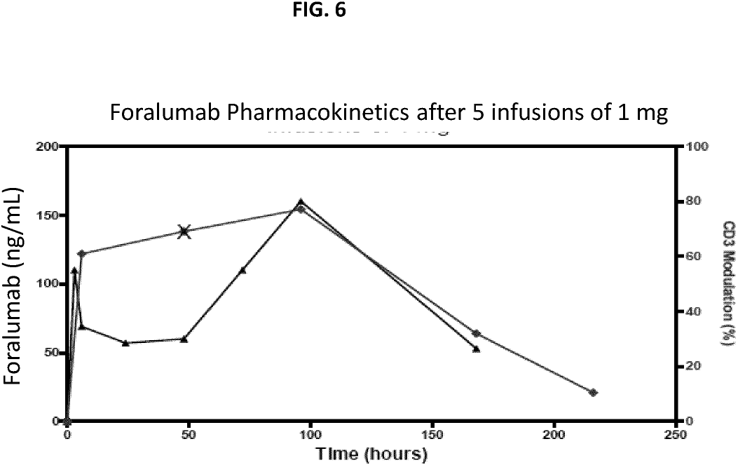

Exposure based on AUC0-t from Study Day 1 to 5 indicated there was accumulation of foralumab in the blood plasma. One subject (030-0003 in FIG. 4) who was exposed to 5 doses of 1.0 mg foralumab corresponding to approximately 21 µg/kg/dose (+/- 500 µg/m2/dose) had plasma drug concentrations increasing during the treatment period (FIG. 6). When overlayed, the PK and PD profiles in this subject correlated, both showing a peak at the end of the treatment period as shown in the graph below (FIG. 6). This may be due to the frequency of dosing and/or the depletion of the target having a decreased effect on mAb disposition.

Overall these observations suggest that intravenous administration of foralumab at doses of 1.0 mg (i.e. 21 µg/kg or 500 µg/m2) and above may result in an accumulation of drug over the 5 days dosing period. However, drug is expected to be eliminated rapidly and within approximately 3-4 days of the final dose.

### Pharmacodynamic Results

There was no discrimination by foralumab dose on its expected pharmacology for all pharmacodynamic analyses. All dose levels of foralumab had effects on the TCR- CD3 complex and cellular populations in the time courses observed.

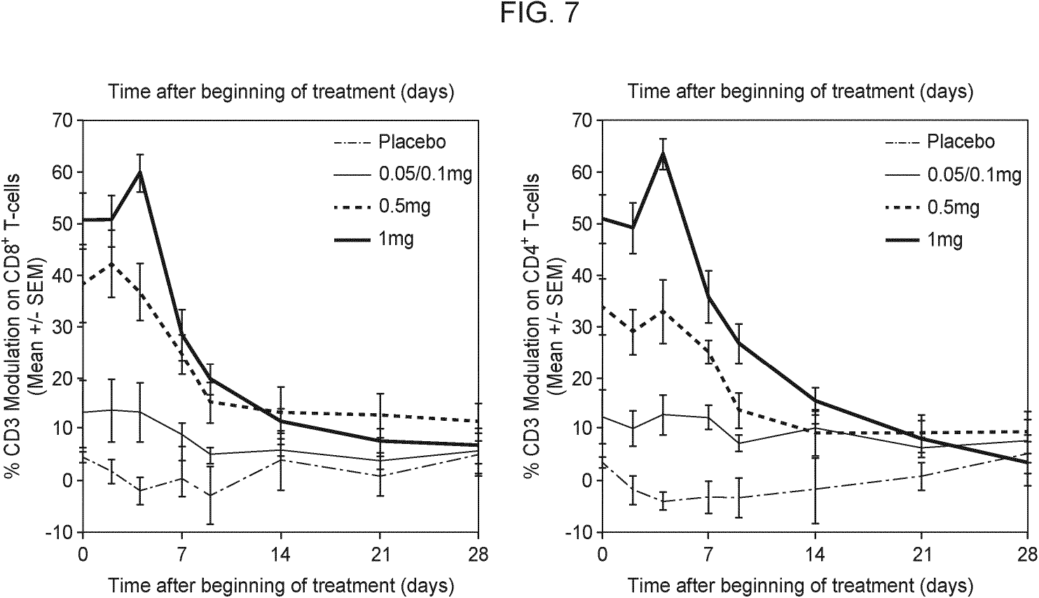

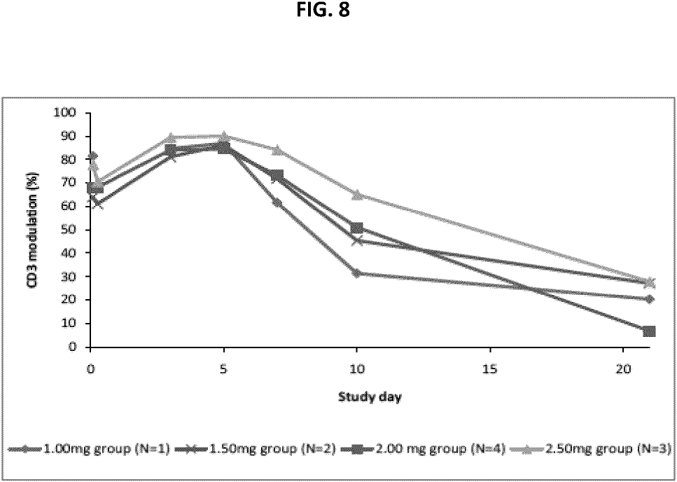

Modulation of the TCR-CD3 complex was measured in CD8+ or CD4+ T cells after the beginning of treatment at the indicated time points (FIG. 7) The maximum modulation of TCR-CD3 complex for all treatment groups was observed at the end of the treatment period on Study Day 5. The mean modulation across all treatment groups on Study Day 5 was 81.1% and the highest mean TCR-CD3 complex modulation at this point was observed in treatment cohort 8 (94%) (FIG. 8). TCR-CD3 modulation gradually decreased after the treatment period ended (FIG. 8). All patients in all treatment cohorts (for whom CD3 modulation data were available) achieved CD3 modulation above 50% (FIG. 8). CD3 modulation across all treatment groups remained above 50% for a mean duration of 8.7 days and above 30% for a mean duration of 12.9 days.

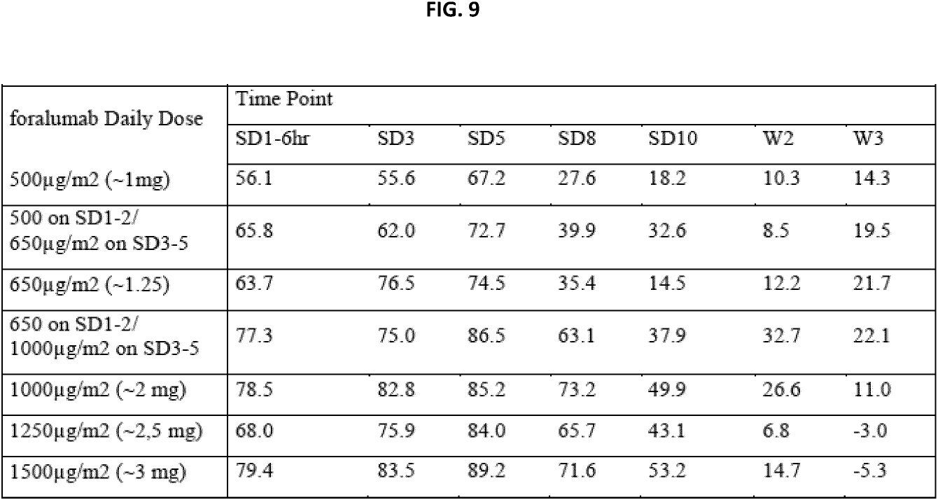

A preliminary kinetic profile of TCR-CD3 modulation per cohort (doses from 500 to 1500 µg/m2) of 3 patients is summarized in FIG. 9. The profile describes a dose dependent effect with a peak reached at day 5 followed by a gradual decline until day 21 (Week 3). The 3 highest dose regimen groups currently tested and ranging between 2 and 3 mg/day have the same profile with a mean TCR-CD3 modulation at day 10 around 50%, representing a plateau reached at those dose regimens (FIG. 9).

Circulating leucocytes and sub-population counts were generated during and after the time course of foralumab dosing. There was a transient elevation in CD45+ leucocyte count in all the foralumab treatment cohorts (overall mean 68.7% elevation) at 6 hours post-dose on Study Day 1 followed by a return to levels close to, or below baseline in most cohorts. By Week 3 the CD45+ leucocyte counts were below their baseline value in all cohorts except for 1 and 4. There was a rapid and almost complete disappearance (>90%) of CD45+ lymphocytes, CD3+ T-cells, CD3+CD4+ T-cells (helper T-cells) and CD3+CD8+ T-cells (cytotoxic T-cells) from the circulation within 24 h of first infusion in all treatment groups followed by a return close to baseline values by Week 3. These results strongly suggest foralumab can induce lymphodepletion in humans. There was no apparent dose-response in the reduction or recovery although the recovery in cohort 1 was faster than in all other cohorts, except for CD8+ cell counts. There was also a rapid decrease in CD3-CD19+ (B-cell) and CD3-CD16+CD56+ cell (Natural killer cell) counts in all treatment groups 6-hours post-dose on Study Day 1, with a variable (non-dose-dependent) recovery in these cell counts at Study Day 3 and values above baseline at week 3 in the majority of cases.

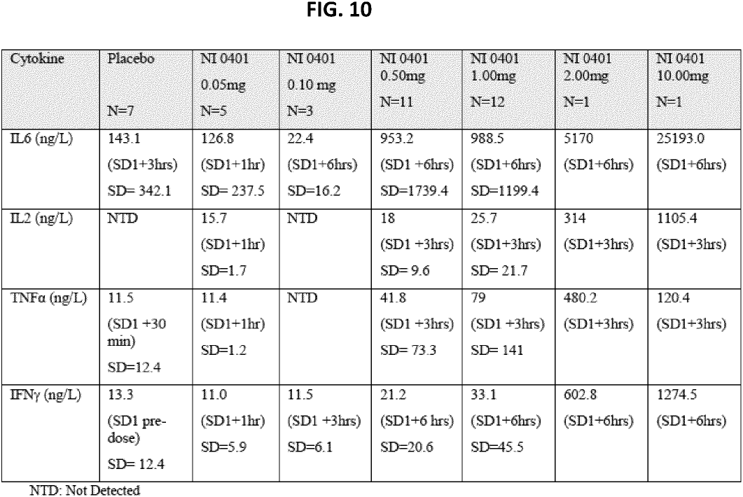

Cytokine levels were assessed in subjects administered foralumab (FIG. 10). Substantial variations between subjects in their release of cytokines following treatment with foralumab were observed, which did not appear to be dose-related. In patients with notable elevations of pro-inflammatory cytokines, this was accompanied by symptoms suggestive of an infusion-related reaction (IRR), although most symptoms were mild and of short duration. Most patients had little or no evidence of pro-inflammatory cytokine release on subsequent treatment days.

### Safety Results

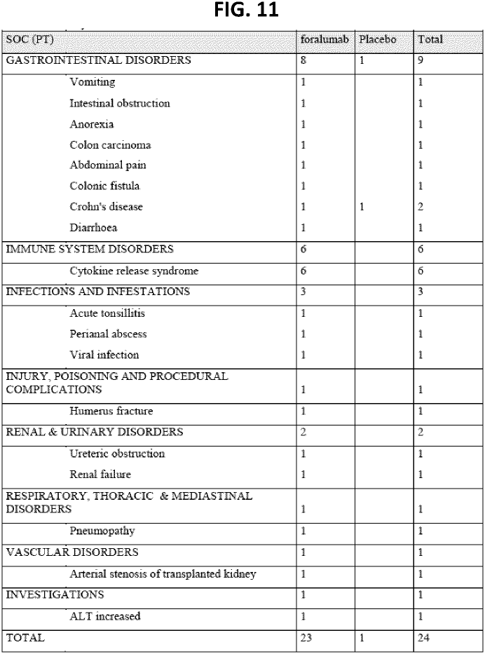

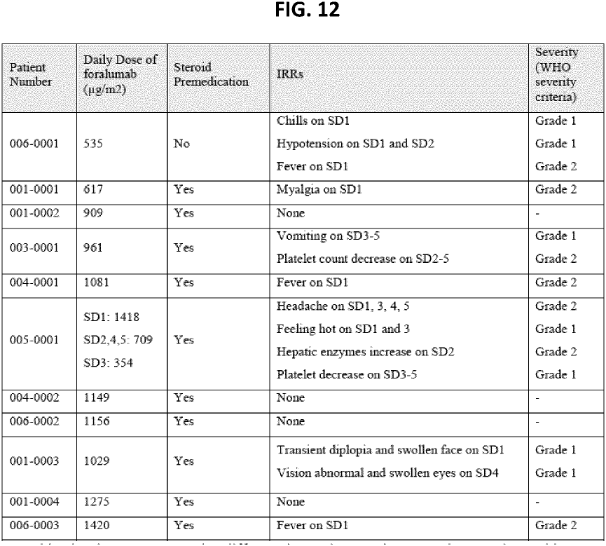

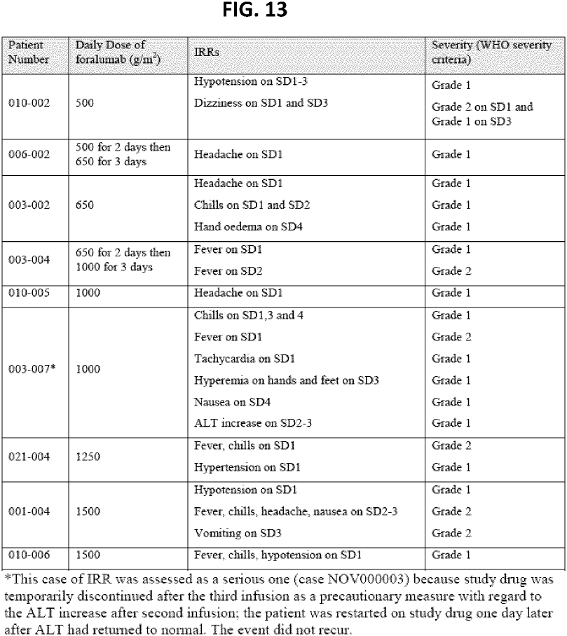

Adverse Events (AEs) were assessed in human subjects given foralumab by intravenous administration (FIG. 11). Nineteen of the 24 patients (79%) experienced a total of 94 AEs of which 3 (3%) were serious adverse events (SAEs) SAEs. Fifty-eight AEs (62%) were of mild severity, 32 (34%) of moderate severity and 4 (4.3%) were severe. Sixty-eight out of the 91 non-serious AEs (75%) were considered by the investigators to have a reasonable possibility of being drug-related. The AEs that occurred during the 5-day treatment period were mainly defined as being infusion related reactions (IRRs) (61%) as they were reported during, or within 24 hours following, an infusion of foralumab (FIG. 12 and FIG. 13). The most common IRRs were chills (9 events in 6 patients), pyrexia (8 events in 7 patients), headache (8 events in 6 patients), hypotension (5 events in 3 patients) and elevated ALT (3 events in 3 patients).

One IRR was reported as an SAE: A patient in cohort 5 had transient elevation of ALT on Study Day 2 leading to interruption in study drug treatment. Visual inspection indicates an apparent dose-response in the reporting of treatment-related AEs, with more drug-related AEs reported by the higher dose cohorts; all the blood and lymphatic system disorders were reported by the two highest dose cohorts.

Three SAEs were reported; two were considered to be unrelated to the study drug: one patient had a relapse of Crohn’s disease 8 days after completion of the 5-day treatment, resulting in prolongation of hospitalisation and a second patient had a multi-fragmental fracture of the left humeral bone with bone fragments displacement following a fall in the street, one and a half months after the end of study treatment. One SAE was considered to be related to the study drug: a transient elevation of ALT on Study Day 2 leading to interruption in study drug treatment. Few AEs (18) were reported after the 5-day treatment period during the study. There were no deaths and no AEs that led to study discontinuation.

Hematology and biochemical analysis in subjects receiving foralumab indicated that, in general, mean hemoglobin, hematocrit and red blood cell count values remained stable in all treatment cohorts over time and there was no substantial change over time in mean platelet count across the treatment cohorts. By Study Day 5 there was a reduction from baseline in mean total white blood cell count of -2.98 10E9/L across all treatment cohorts, with no evidence of a dose-response. White blood cell counts had recovered by Week 12. Mean neutrophil and monocyte counts showed the same pattern, with a mean drop on Study Day 5 and recovery by Week 12 and Week 3 respectively.

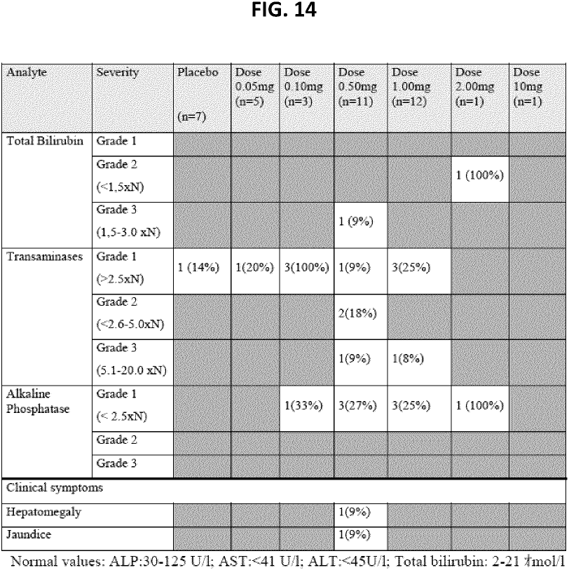

Liver function was assessed in subjects receiving foralumab and some transient and non-serious liver test abnormalities were detected (FIG. 14). Five patients (15%) had isolated and transient rise in ALP above the upper limit of normal range starting at day 5 in two cases, and at week 2, 3 and 4 in the other cases respectively. One patient had preexisting abnormal ALP levels. One patient had a transient and isolated rise in AST /ALT at 3.5 twice the upper limit of normal range at day 5 which was normalized at week 2. Six patients (18%) had liver tests abnormalities depicting a cholestatic liver injury of mild severity and transient in duration. No rise in serum bilirubin was associated except in one patient. Out of these six patients, three already had preexisting liver test abnormalities of same magnitude. The rise of hepatic enzymes occurred mainly at day 5 and returned to basal levels within one week. One patient had mild jaundice (rise in bilirubin on day 2 and resolved on day 4) and another had hepatomegaly (a second rise in liver enzymes reoccurred at week 4 in this patient who also had preexisting liver test abnormalities). No other signs of hepatic insufficiency were noted in these cases and no causal factor has been identified.

### Conclusions

From the safety data it can be concluded that the dose-limited-toxicity dose was not reached in these studies. However, the safety profile of foralumab was extended by the steroid premedication from a daily dose of 500 mg/m2 (~1.00 mg) to 1750 mg/m2 (~3.5 mg). This study has shown foralumab to be pharmacologically active. Foralumab had an impact on the TCR-CD3 complex and on T-cell subsets reflecting the expected pharmacology of this drug and its target. After foralumab treatment, lymphocyte counts were decreased below the normal range in all patients. There was no obvious effect of foralumab dose on the duration of lymphocyte depletion. Mean lymphocyte counts were back close to pre-treatment values by Week 4. Lymphocyte depletion is an expected effect of administration of an anti-CD3 antibody.

### OTHER EMBODIMENTS

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1. A method of improving cell expansion and/or survival comprising contacting a cell with a composition comprising an anti-CD3 antibody.

2. The method of claim 1, wherein the cell is an engineered cell.

3. The method of claim 1, wherein the cell is a lymphocyte.

4. The method of claim 3, wherein the lymphocyte is a B-cell or a T-cell.

5. The method of claim 4, wherein the T cell is a CAR-T cell.

6. The method of claim 1, wherein the cell is a stem cell.

7. The method of claim 6 wherein the stem cell is a human embryonic stem cell, a tissue-specific stem cell, a neural stem cell, a mesenchymal stem cell, a hematopoietic stem cells, an induced pluripotent stem cell, an epidermal stem cell, an epithelial stem cell, and/or a neural stem cell.

8. The method of any one of the precedingclaims, wherein the contacting is ex vivo, in vivo or both.

9. A method of enhancing cell therapy in a subject comprising administering to a subject in need thereof a composition comprising an anti-CD3 antibody.

10. The method of claim 9, wherein the cell therapy is CAR-T cell therapy.

11. The method of claim 9, where in the cell therapy is stem cell therapy.

12. The method of claim 11, wherein the stem cell is a human embryonic stem cell, a tissue-specific stem cell, a neural stem cell, a mesenchymal stem cell, a hematopoietic stem cells, an induced pluripotent stem cell, an epidermal stem cell, an epithelial stem cell, and/or a neural stem cell.

13. The method of any one of the precedingclaims, wherein the composition improves the clinical outcome of the cell therapy.

14. The method of any one of the preceding claims, wherein the anti-CD3 antibody is a monoclonal antibody, a bispecific antibody or a trispecific antibody.

15. The method of claim 14, wherein the bispecific antibody has specificity for CD3 and IL-6R, CD28 or TNF.

16. The method of claim 14, wherein the trispecific antibody has specificity for:
i. CD3, IL6R, and CD28;
ii. CD3, IL6R and TNF;
iii. CD3, CD28 and TNF; or
iv. IL-6, IL-17.

17. The method of any one of the preceding claims, wherein the composition further include one or more co-stimulatory agents.

18. The method of claim 17, wherein the co-stimulatory agent is a CD28 antibody, an IL-6R antibody, a PI3K inhibitor, an Akt inhibitor or a mTor inhibitor.

19. The method of claim 18, wherein the CD28 antibody is a monoclonal antibody, a bispecific antibody or a trispecific antibody.

20. The method of claim 19, wherein the bispecific antibody has specificity for CD28 and IL-6R or TNF.

21. The method of claim 19, wherein the trispecific antibody has specificity for CD28, IL-6R, and TNF.

22. The method of claim 18, wherein the IL-6R antibody is a monoclonal antibody, a bispecific antibody or a trispecific antibody.

23. The method of claim 22, wherein the bispecific antibody has specificity for IL-6R and CD28 or TNF.

24. The method of claim 22, wherein the trispecific antibody has specificity for IL-6R, CD28, and TNF.

25. The method of any of any one of the preceding claims wherein the CD3 antibody and or the CD28 antibody are coated on macroporous beads.

26. The method of any one of the preceding claims, wherein the CD3 antibody is administered nasally, orally, subcutaneously, intravenously or by inhalation.

27. The method of any one of the preceding claims, wherein the CD3 antibody comprises a heavy chain complementarity determining region 1 (CDRH1) comprising the amino acid sequence GYGMH (SEQ ID NO: 42), a heavy chain complementarity determining region 2 (CDRH2) comprising the amino acid sequence VIWYDGSKKYYVDSVKG (SEQ ID NO: 43), a heavy chain complementarity determining region 3 (CDRH3) comprising the amino acid sequence QMGYWHFDL (SEQ ID NO: 44), a light chain complementarity determining region 1 (CDRL1) comprising the amino acid sequence RASQSVSSYLA (SEQ ID NO: 45), a light chain complementarity determining region 2 (CDRL2) comprising the amino acid sequence DASNRAT (SEQ ID NO: 46), and a light chain complementarity determining region 3 (CDRL3) comprising the amino acid sequence QQRSNWPPLT (SEQ ID NO: 47).

28. The method of claim 27, wherein the CD3 antibody comprises a variable heavy chain amino acid sequence comprising the amino acid sequence of SEQ ID NO: 48 and a variable light chain amino acid sequence comprising the amino acid sequence of SEQ ID NO: 49.

29. The method of claim 27, wherein the CD3 antibody comprises a comprising a heavy chain amino acid sequence comprising the amino acid sequence of SEQ ID NO: 50 and a light chain amino acid sequence comprising the amino acid sequence of SEQ ID NO: 51.

30. The method of claim 22, wherein the IL-6R antibody comprising a VH CDR1 region comprising the amino acid sequence of SEQ ID NO: 15, a VH CDR2 region comprising the amino acid sequence of SEQ ID NO: 37, a VH CDR3 region comprising the amino acid sequence of SEQ ID NO: 35, a VL CDR1 region comprising the amino acid sequence of SEQ ID NO: 24, a VL CDR2 region comprising the amino acid sequence of SEQ ID NO: 25, and a VL CDR3 region comprising the amino acid sequence of SEQ ID NO: 26.

31. The method of claim 22, wherein the IL-6R antibody is tocilizumab or sarilumab.

32. The method of any one of claims 9 to 31, wherein the anti-CD3 antibody is administered prior to, subsequent to, both prior and subsequence to, and/or simultaneously with administration of a cell therapy cell composition to the subject.

33. The method of any one of claim 32, wherein the anti-CD3 antibody is administered 24 to 48 hours prior to administering the cell therapy composition to the subject.

34. The method of claims 32 or 33, wherein administering an anti-CD3 antibody prior to administering the cell therapy composition results in lymphodepletion and/or immunosuppression.

35. The method of claim 32, wherein the anti-CD3 antibody is administered 14 to 21 days after administering the cell therapy cell composition.

36. The method of any one of the preceding claims, wherein the anti-CD3 antibody is formulated in a pharmaceutical composition comprising the anti-CD3 antibody and a pharmaceutically acceptable carrier.

37. The method of claim 36, wherein the pharmaceutical composition comprises a unit dose of 0.5 mg, 1.0 mg, 1.5 mg, 2.0 mg, 2.5 mg, 3.0 mg, or 4 mg of anti-CD3 antibody.
